SEQUENCE LISTING (19 sequences)

SEQ ID NO: 1 - DNA, 40 bp, Artificial Sequence
  Synthetic oligonucleotide
tatcatcaaagccatgaacaaagcagccgcgctggatgaa

SEQ ID NO: 2 - DNA, 20 bp, Artificial Sequence
  Synthetic oligonucleotide
tatcatcaaagccatgaaca

SEQ ID NO: 3 - DNA, 20 bp, Artificial Sequence
  Synthetic oligonucleotide
tatattcactcagcaacccc

SEQ ID NO: 4 - DNA, 40 bp, Artificial Sequence
  Synthetic oligonucleotide
gagttcgtgtccgtacaactggcgtaatcatggcccttcg

SEQ ID NO: 5 - DNA, 20 bp, Artificial Sequence
  Synthetic oligonucleotide
gagttcgtgtccgtacaact

SEQ ID NO: 6 - DNA, 20 bp, Artificial Sequence
  Synthetic oligonucleotide
gactcctccacagagaaaca

SEQ ID NO: 7 - DNA, 20 bp, Artificial Sequence
  Synthetic oligonucleotide
taatacgactcactataggg

SEQ ID NO: 8 - DNA, 20 bp, Artificial Sequence
  Synthetic oligonucleotide
tccctttagtgagggttaat

SEQ ID NO: 9 - DNA, 20 bp, Artificial Sequence
  Synthetic oligonucleotide
attaaccctcactaaaggga

SEQ ID NO: 10 - DNA, 24 bp, Artificial Sequence
  Synthetic oligonucleotide
attggtaaattggtaaatgaattg

SEQ ID NO: 11 - DNA, 64 bp, Artificial Sequence
  Synthetic oligonucleotide
taatacgactcactatagggcaattcatttaccaatttaccaatactcctgcctgagtttccag

SEQ ID NO: 12 - DNA, 24 bp, Artificial Sequence
  Synthetic oligonucleotide
aaagttgagtattgatttgaaaag

SEQ ID NO: 13 - DNA, 64 bp, Artificial Sequence
  Synthetic oligonucleotide
taatacgactcactatagggcttttcaaatcaatactcaactttcagaaactgagctccgggtg

SEQ ID NO: 14 - DNA, 24 bp, Artificial Sequence
  Synthetic oligonucleotide
aaagtgaaaaagattgattgatga

SEQ ID NO: 15 - DNA, 64 bp, Artificial Sequence
  Synthetic oligonucleotide
taatacgactcactatagggtcatcaatcaatctttttcacttttccttaggttgatgtgcttg

SEQ ID NO: 16 - DNA, 24 bp, Artificial Sequence
  Synthetic oligonucleotide
aaagaaagattgttgagattatga

SEQ ID NO: 17 - DNA, 64 bp, Artificial Sequence
  Synthetic oligonucleotide
taatacgactcactatagggtcataatctcaacaatctttcttttctggcgttccacctccaag

SEQ ID NO: 18 - DNA, 24 bp, Artificial Sequence
  Synthetic oligonucleotide
ttagtgtagtaagtttaaagtgta

SEQ ID NO: 19 - DNA, 64 bp, Artificial Sequence
  Synthetic oligonucleotide
taatacgactcactatagggtacactttaaacttactacactaaccctggaccaccagccccag

(19) United States
(12) Patent Application Publication
(10) Pub. No.: US 2011/0136104 A1
(43) Pub. Date: Jun. 9, 2011

(54) MULTIPLEXED QUANTITATIVE PCR END POINT ANALYSIS OF NUCLEIC ACID TARGETS

(75) Inventors: Daniel Colin Pregibon, Cambridge, MA (US); Patrick Seamus Doyle, Boston, MA (US)

(73) Assignee: Massachusetts Institute of Technology, Cambridge, MA (US)

(21) Appl. No.: 12/631,322

(22) Filed: Dec. 4, 2009

(51) Int. Cl. C12Q 1/68; C07H 21/04; C12M 1/34

(52) U.S. Cl. 435/6; 536/24.3; 435/287.2; 977/774

(57) ABSTRACT

Certain embodiments of the present invention are directed to one pot multiplexed quantitative PCR methods for end point analysis of a plurality of nucleic acid targets in a complex sample without user intervention, and to various encoded particles on which are immobilized one or more probes that hybridize with the plurality of targets. Certain other embodiments are directed to a new “multiple-color genetic variation detection method” that can detect SNPs and kit using one chamber multiplexed endpoint PCR and differentially labeled allele-specific primers (one recognizing only the wild type allele and one only the mutant allele).

### BACKGROUND OF THE INVENTION

1. Field of the Invention

This invention generally relates to methods for performing quantitative multiplexed endpoint PCR in a single chamber to amplify, detect and quantify the amount of one or more target nucleic acids in a complex sample using encoded polymer particles on which are immobilized probes that are complementary to the targets.

2. Description of the Related Art

The accurate detection of nucleic acid targets, including SNP detection, is of utmost importance for clinical diagnostics, drug discovery, and basic science research. Ideally multiple targets can be detected in a single assay with high-throughput analysis, high sensitivity, specificity between closely related targets, and a wide dynamic range. Real-time polymerase chain reaction, also called quantitative real time polymerase chain reaction (QRT-PCR) or kinetic polymerase chain reaction, is used to amplify and simultaneously quantify one or more targeted DNA or RNA molecules. Recently, methods have been described that permit multiplexed real-time PCR in a single chamber by combining a complex sample, primers, the required PCR cocktail and enzymes with particles on which are immobilized probes that are complementary to targeted nucleic acids. Whitman et al. US Application Serial No. 2008/0305481. However, there is still a need for approaches that do not require multiple analysis steps for quantification and those that are optimized for efficient detection of genetic variability, particularly single nucleotide polymorphisms.

### SUMMARY OF THE INVENTION

Certain embodiments of the invention are directed to a method of amplifying and quantifying a plurality of nucleic acid targets in a sample having the steps of: a) combining in a chamber: the sample comprising the plurality of nucleic acid targets; a labeling agent, a plurality of primer pairs for priming amplification of the plurality of nucleic acid targets, wherein the primer pairs hybridize to the targets at a primer annealing temperature, a plurality of particles on which are immobilized a plurality of nucleic acid probes that are complementary to the plurality of nucleic acid targets, and a PCR cocktail containing enzymes for amplifying the nucleic acid targets; b.) performing one or more amplification cycles to form labeled amplification products for each of the plurality of nucleic acid targets, c.) hybridizing the labeled amplification products for each of the plurality of nucleic acid targets to the respective complementary probes at a hybridization temperature that is at least from about 2-15° C. higher than the primer annealing temperature but lower than a Tm of target-probe complexes; d.) detecting and quantifying a signal from the labeled amplification products for each of the plurality of nucleic acid targets hybridized to the respective complementary probes; and e.) comparing the quantified amplification product signal for each of the nucleic acid targets to a signal from a known amount of a known reference nucleic acid to quantify the amount of each nucleic acid target in the sample. The known reference nucleic acid of step e) is an endogenous reference gene (such as nucleolar RNA, beta-actin, GADPH or 18S RNA), or an external nucleic acid added to the sample, or the nucleic acid target known amounts of which are plotted on a standard curve. In these embodiments the labeling agent binds to one primer of each of the primer pairs. In other embodiments the mixture of step a further includes a free probe that is complementary to a region of the amplification product and wherein the free probe is bound to the labeling agent such as a fluorescent label selected from the group comprising 6-FAM™, Alexa Fluor, Fluorescein, Phycoerythrin, Cy3, Cy5, Cy5.5, Dy 750, HEX™, Iowa Black®, IRDye®, Joe, LightCycler 640, MAX 550, Rhodamine Green™, Rhodamine Red™, ROX™, TET™, TEX 615, Texas Red®, TYE (including TYE™ 563, TYE™ 665, TYE™ 705), WellRED™ D2, WellRED™ D3, WellRED™ D4 and TAMRA dyes. In certain embodiments the probe includes a 10 base pair to about 50 base pair sequence that is complementary to the target nucleic acid sequence and the 3′ end of the probe includes a blocked 3′ hydroxyl group such as a phosphate group or a 3′ inverted dT or dideoxycytidine modification. In certain embodiments the probes include a locked nucleic acid (LNA) modification.

In certain embodiments the primer annealing temperature is from about 35° C. to about 60° C. and the hybridization temperature is from about 37 to about 75° C. In other embodiments the primers are from about 10 to about 25 base pairs in length and the amplification products are from about 50 to about 100 base pairs long, preferably from about 50 to about 70 base pairs long. In some embodiments of the method the primers have a melting temperature of about 35° C. to about 65° C. and the probe-amplicon complexes have a melting temperature of about 40 to about 75° C. In certain embodiments the hybridization step (c) is from about 20 minutes to about 90 minutes long and from about 20 to about 40 amplification cycles are performed in step (b).

In other embodiments the particles are encoded polymer particles preferably hydrogels of polyethylene glycol, that include one or more different probes that are either immobilized on the particle or incorporated into the particle substrate. The particles can be encoded using fluorophores, chromophores, graphical codes, radio frequencies, magnetic properties, radioactivity, or diffractive gratings, and they can be composed of polymer, glass, silica, or metal.

An embodiment is directed to a method of amplifying and detecting genetic variation such as an SNP at a known mutation site in a gene in a sample comprising a plurality of nucleic acid targets having the steps of: a) combining in a chamber: a first primer pair for priming amplification of a first allele of the gene using multiplex PCR, wherein the primers hybridize to the targets at a primer annealing temperature, and wherein one primer of the first primer pair is labeled with a first reporter, and a second primer pair for priming amplification of a second allele of the gene using multiplex PCR, wherein the primers hybridize to the targets at a primer annealing temperature, and wherein one primer of the first primer pair is labeled with a second reporter, a plurality of particles on which are immobilized a plurality of nucleic acid probes that are complementary to a nucleic acid sequence that is common to both the first and second alleles, wherein the common sequence is adjacent to the known mutation site, and a PCR cocktail containing enzymes for amplifying nucleic acid targets, b) performing one or more amplification cycles to form labeled amplification products for the first and second alleles, c) hybridizing the labeled amplification products to the probes at a hybridization temperature that is at least about 2° C. to 15° C. degrees higher than the primer annealing temperature, and (d) detecting a signal from the reporters on the labeled amplification products hybridized to the probes on the particles, and comparing the two signals thereby detecting the relative quantities of the first and the second alleles on the particle. The reporter can be any reporter known in the art including fluorophores, quantum dots, and radioisotopes. Preferably the primer labeled with the first reporter includes a nucleotide at the 3′ end that is complementary to the nucleotide in the first allele at the known mutation site, and the primer labeled with the second reporter includes a nucleotide at the 3′ end that is complementary to the nucleotide in the second allele at the known mutation site, and the nucleic acid probe that is complementary to a nucleic acid sequence that is common to two different alleles of a particular gene, for example the sequence is adjacent to a known SNP mutation site on the gene. Certain embodiments are directed to this probe, and other embodiments are directed to a particle for nucleic acid detection, that includes the probe.

Other embodiments are directed to kits for detecting genetic variation at a known mutation site in a gene, that include a first allele-specific primer pair for priming amplification of a first allele of the gene, wherein the primer that is extended to form the amplification product is labeled with a first reporter, and a second allele-specific primer pair for priming amplification of a second allele of the gene, wherein the primer that is extended to form the amplification product is labeled with a second reporter, and encoded hydrogel particles on which are immobilized a plurality of nucleic acid probes that are complementary to a nucleic acid sequence that is common to both the first and second alleles, wherein the common sequence is adjacent to the known mutation site.

Other embodiments are directed to a cartridge for use in dedicated equipment that performs multiplexed end-point quantitative PCR according to the steps of claim 1, and to portable devices that incorporate the cartridge.

### BRIEF DESCRIPTION OF THE DRAWINGS

The present invention is illustrated by way of example, and not by way of limitation, in the figures of the accompanying drawings and in which:

FIG. 1: Encoded particle assay limits of detection vs. time. 1A: Sensitivities were identified as the point where the measured signal-to-noise ratio was 3, and were plotted against a quantitative model. FIG. 1B: Specificity was confirmed using four microRNA let-7 family members. Let-7a synthetic target was spiked into E. coli RNA together with particles bearing probe regions complementary to let7a, 7b, 7c, and 7d, respectively, which have sequences differing by only one or two nucleotides.

FIG. 2: Demonstration of thermal stability of encoded particles and proof of no-rinse assay. Hydrogel particles bearing two probe regions, a negative control region, and barcode region were heated at 95° C. for 20 min. They were then incubated with 500 attomole target at 37° C. for 60 minutes and imaged immediately afterward. No rinsing steps were used prior to scanning to detect fluorescence. Scale bar is 50 μm.

FIG. 3: Multiplexed End Point-PCR for multiplexed detection of nucleic acid targets from lambda phage. Fluorescence images of particles after PCR. Shown is a schematic of the particle design, and an image of fluorescence on scanned particles hybridized to labeled amplicons from two samples containing lambda phage DNA and either primers for target #1 or target #2.

FIG. 4: Multiplexed PCR detection and quantification of mRNA amplicons. Four different mRNA targets and an internal reference were reverse-transcribed and amplified using RT-PCR. Targets detected with multiplexed encoded hydrogel particles (“Barcoded particles”) compared to Luminex particles.

FIG. 5: Schematic of a method for amplifying and detecting pathogens in a complex nucleic acid sample.

FIG. 6. Primer and particle design for allele-specific SNP amplification and detection using two colors. Two primers, one specific for each allele, are differentially labeled with unique fluorophores (Cy3 and Cy5) to selectively label allele-specific amplicons. Encoded particles have a single probe region to capture either allele amplicon, with a probe designed to give a complex Tm of ˜70° C. Hybridization will be carried out at 60° C. and particles will be scanned using two-color detection. P=phosphorylation of the 3′ end.

### DEFINITIONS

Allele-specific PCR is a technique that is used to identify point mutations called single-nucleotide polymorphisms (SNPs) (single base differences in DNA). It requires prior knowledge of a DNA sequence, including differences between alleles, and uses primers whose 3′ ends encompass the point mutation.

Asymmetric PCR means the preferential amplification of one strand of the original DNA more than the other. PCR is carried out as usual, but with a great excess of the primers for the chosen strand.

Annealing, in genetics, means for DNA or RNA to pair by hydrogen bonds to a complementary sequence, forming a double-stranded polynucleotide. The term is often used to describe the binding of a DNA probe or of a primer to a DNA strand during a polymerase chain reaction (PCR).

The terms “amplicons” and “amplification products” are used interchangeably.

Encoded Particle (EP) means a polymer particle, preferably a hydrogel, which is labeled with an identifying characteristic (one or multiple fluorescent or features, a graphical pattern, magnetic properties, a radio frequency tag, etc.) and has a nucleic acid probe immobilized in it. Encoded particles preferably have a fluorescently labeled coded region and at least one distinct probe region comprising a plurality of nucleic acid probes that hybridize to a particular target nucleic acid. The encoded particles can have more than one distinct probe region that are each specific for a different particular target nucleic acid. In an embodiment the encoded particles also has an inert region that is not labeled, separating the coded region from the first flanking probe region.

Hybridization temperature as used herein means the temperature at which the PCR hybridization step is conducted during which step the PCR-generated amplification products hybridize to the corresponding complementary probes immobilized on the particles.

Locked nucleic acid or “LNA” means a bi-cyclic compound that is structurally similar to RNA nucleosides. LNAs have a furanose conformation that is restricted by a methylene linker that connects the 2′-O position to the 4′-C position. Locked nucleic acids can increase complex stability approximately tenfold and can alter the hybridization temperature of a nucleic acid to a probe.

Melting Temperature (Tₘ) by definition means the temperature at which one half of a DNA duplex will dissociate to become single stranded when two nucleic acid species are present at equal levels, and it indicates duplex stability.

Multiplex-PCR means the use of multiple, unique primer sets within a single PCR mixture to produce amplicons of varying sizes specific to different DNA sequences.

Multiplexed End Point Quantitative PCR means a one pot multiplexed PCR method for amplification and end point detection and quantification of multiple nucleic acid targets in a single sample, wherein a complex sample is combined with primer sets unique for each targeted nucleic acid, PCR cocktail, enzymes, labeling agent (which can be free or attached to a primer for example) and a plurality of particles, preferably encoded polymer particles, on which are immobilized a plurality of probes complementary to each respective nucleic acid targets. The method has only one cycle of amplification followed by a single hybridization step, preferably at a hybridization temperature that is at least about 2 to about 15° C. higher than the primer annealing temperature. After the hybridization step, the signal from the labeled amplification product bound to the corresponding complementary probes on the particle is detected and quantified. The amplification product signal is then compared to either 1) a standard curve that correlates the signal generated by binding of a nucleic acid target to the corresponding probe over a broad range of target concentrations, 2) an endogenous reference gene or 3) an external target that is spiked into the sample at a known concentration to quantitate the amount of nucleic acid target in the complex sample.

PCR cocktail as used herein means a solution for PCR including: deoxynucleotide triphosphates (dNTPs); a buffer solution providing a suitable chemical environment for optimum activity and stability of the DNA polymerase and/or reverse transcriptase; divalent cations, typically magnesium ions (Mg²+); and monovalent cation potassium ions.

Primer annealing temperature means the temperature at which a primer anneals to a DNA target that is to be amplified. The primer melting temperature is the estimate of the DNA-DNA hybrid stability and it is critical in determining the annealing temperature.

Reverse Transcription PCR (RT-PCR) means a method used to amplify, isolate or identify a known RNA sequence from a sample such as cellular or tissue RNA. The PCR is preceded by a reaction using reverse transcriptase to convert RNA to cDNA. RT-PCR is widely used in expression profiling, to determine the expression of a gene or to identify the sequence of an RNA transcript, including transcription start and termination sites. The present methods are suitable for RT-PCR of RNA targets.

### DETAILED DESCRIPTION

Certain embodiments of the present invention are directed to one pot multiplexed quantitative PCR methods for end point analysis of a plurality of nucleic acid targets in a complex sample without user intervention. A complex sample is combined with primer sets unique for each targeted nucleic acid, PCR cocktail, enzymes, labeling agent (which can be free or attached to a primer, for example) and a plurality of particles, preferably encoded polymer particles with one or more probe regions on which are immobilized a plurality of probes complementary to each respective nucleic acid target. The method has only one set of amplification cycles followed by a single hybridization step at a hybridization temperature that is significantly higher than the primer annealing temperature, preferably at least about 2 to about 15° C. higher. After the hybridization step, the signal from the labeled amplification products bound to the corresponding complementary probes on the particle is detected and quantified. Typically about 30 amplification cycles are needed to generate the sufficient amount of labeled amplicons for detection. Any particles on which a nucleic acid probe can be immobilized can be used; however, encoded polymer particles made with a porous hydrogel matrix in which the probes are dispersed are preferred. These particles have an encoded region and one or more distinct probe regions in which are immobilized a plurality of probes that are complementary to a particular target nucleic acid. In an embodiment encoded polymer particles are analyzed in a rapid flow-through scanning device, wherein each particle is directly imaged using a fluorescence microscope and a camera.

Certain other embodiments are directed to a new “multiple-color genetic variation detection method” that can detect SNPs and kit using one chamber multiplexed endpoint PCR and differentially labeled allele-specific primers (one recognizing only the wild type allele and one only the mutant allele). The primers can be differentially labeled with unique fluorophores (for example Cy3 and Cy5). The primers are only extended by polymerase if the sequence at the 3′ end of the primer shows perfect complementary to the target strand. The differentially labeled wild type or mutant amplicons formed by extension of the respective labeled primers are each capable of binding to a probe that is complementary to a region of the allele immediately adjacent to the mutation site that is common to both the wild type and mutant alleles. The color of the amplicon bound to the probes on the particle identifies the allele, mutant or wild type that was in the sample.

An embodiment of the invention is directed to a method of amplifying and quantifying a plurality of nucleic acid targets in a sample having the steps of:
a. combining in a chamber:
the sample comprising the plurality of nucleic acid targets;
a labeling agent,
a plurality of primer pairs for priming amplification of the plurality of nucleic acid targets, wherein the primer pairs hybridize to the targets at a primer annealing temperature,
a plurality of particles on which are immobilized a plurality of nucleic acid probes that are complementary to the plurality of nucleic acid targets,
and a PCR cocktail containing enzymes for amplifying the nucleic acid targets;
b. performing one or more amplification cycles to form labeled amplification products for each of the plurality of nucleic acid targets,
c. hybridizing the labeled amplification products for each of the plurality of nucleic acid targets to the respective complementary probes at a hybridization temperature that is at least from about 2-15° C. higher than the primer annealing temperature but lower than a Tm of target-probe complexes;
d. detecting and quantifying a signal from the labeled amplification products for each of the plurality of nucleic acid targets hybridized to the respective complementary probes; and
e. comparing the quantified amplification product signal for each of the nucleic acid targets to a signal from a known amount of a known reference nucleic acid to quantify the amount of each nucleic acid target in the sample.

If a particular target being tested is not present in the sample, then the amount of that target amplified, detected and quantified will be zero. The amount of nucleic acid in the target can be determined in step e) by comparing the quantified amplicon signal of step d) to the signal generated by amplification of an endogenous reference gene or a known amount of an external nucleic acid added into the sample, or by comparison to a standard curve plotting the signal generated by binding of the labeled nucleic acid target to a complementary probe over a broad range of target concentrations.

Labeled amplicons can be obtained using any method known in the art for PCR. In a preferred embodiment the labeling agent is bound to one primer of each of the primer pairs. A preferred method uses direct hybridization of labeled primers onto target nucleic acids which generates labeled amplicons that hybridize to complementary probes immobilized on a particle. In another embodiment a two probe system is used in which the labeling agent is bound to a free probe that is complementary to a region of the amplification product. The amplicon binds to less than all of the first probe sequence, leaving a portion of the amplicon unbound and free to bind to the second labeled probe.

Depending on the assay set up, either the sense or antisense strand of the target is labeled by labeling one or the other primer of each primer pair. In an embodiment one of the primers of each pair is prelabeled with a fluorescent label such as 6-FAM™, Alexa Fluor, Fluorescein, Phycoerythrin, Cy3, Cy5, Cy5.5, Dy 750, HEX™, Iowa Black®, IRDye®, Joe, LightCycler™ 640, MAX 550, Rhodamine Green™, Rhodamine Red™, ROX™, TET™, TEX 615™, Texas Red®, TYE™ 563, TYE™ 665, TYE™ 705, WellRED™ D2, WellRED™ D3, WellRED™ D4, TAMRA dyes such as those commercially from AnaSpec™, TYE, or any other suitable fluorophore. In some embodiments indirect labeling of the amplicons is used, such as modifying one primer of each primer pair with a binding moiety such as a biotin group that will bind to a separate labeling agent such as fluorophore-modified streptavadin. Any substance that allows for detection of labeled amplicons may be used and there are many commercially available nucleic acid detection chemistries currently used in real-time PCR. Labeling agents include molecular beacons, DNA binding (intercalating) dyes (such as ethidium bromide, proflavine, daunomycin, doxorubicin, and thalidomide), chromophores, quantum dots, radiolabels, carbon nanotubes, gold nanoparticles, Forster Resonance Energy Transfer (FRET) compounds, fluorophore quencher pairs, labels that interact by Forster Resonance Energy Transfer (FRET) including molecular beacons, binary nucleic acid probes, hydrolysis probes (also known as the TaqMan™ assay (U.S. Pat. No. 5,210,015)), tagged primers, and hybridization probes including two-probe systems. A detailed description of labels and detection chemistries that are suitable for use in the present methods are described in, et al., US 20080305481.

Many methods for polymerase chain reaction (PCR) amplification of DNA targets are known. PCR requires a heat-stable DNA polymerase that preferably has a temperature optimum at around 70° C., such as Taq polymerase. In embodiments where the target is RNA, a heat stable reverse transcriptase is also included in the chamber together with the DNA polymerase. The PCR cocktail further includes: deoxynucleotide triphosphates (dNTPs); a buffer solution providing a suitable chemical environment for optimum activity and stability of the DNA polymerase; divalent cations, typically magnesium ions (Mg²+); and monovalent cation potassium ions. The sample containing the target nucleic acids is added to a chamber containing the DNA polymerase or reverse transcriptase and the PCR cocktail. PCR cocktails are commercially available from various suppliers including New England Biosciences, Sigma-Aldrich, USB, Invitrogen, etc.

As with conventional PCR, the present methods for one-chamber multiplex quantitative PCR with end point detection use thermal cycling to subject the PCR sample to a defined series of temperature steps. After an initial denaturation step, there are a series of amplification cycles, having a denaturation step, followed by an annealing step at a primer annealing temperature, followed by an extension step. Although annealing and extension are typically done at different temperatures, these two steps are optionally done at a common temperature. Typically, the primer annealing temperature is the same as or close to the primer melting temperature.

Ideally, the primers should have as little secondary structure as possible and should be tested for hairpin formation and secondary structures. For multiplexed PCR, compatible primer sets are designed that (1) have similar melting temperatures, (2) do not form hetero-dimers, and (3) are complementary to a region of the target nucleic acid (such as conserved regions of pathogen DNA that are unique for a particular species), and (4) are not complementary to the probe so that they do not prime the DNA probes immobilized on the particles. In a preferred embodiment, the particles are hydrogel particles and the probes are dispersed throughout these porous particles. Primers for use in the new methods typically range from about 10 to about 25 base pairs in length; however, the user may make longer or shorter primers. When encoded hydrogel particles are used, primers and amplicons are preferably short to facilitate rapid hybridization kinetics. For example, primers for hydrogel particles are about 20 base pairs in length or shorter, and amplicons are preferably <100 base pairs, preferably from about 50-70 base pairs in length.

In certain embodiments of the invention, primers may be attached to the particle so that the hybridization probes would not be required since the amplicons would be synthesized on the particles. Typically, only one primer of each primer pair would be attached to the particle. The other primer of the primer pair would be “free floating.”

DNA probes are designed for amplicon capture using any method known in the art. For hybridization to probes immobilized on hydrogel particles, probes typically incorporate about a 20 to 50 base pair region that is complementary to an interior region of the amplicon or the end region of the respective target nucleic acid that overlaps with the non-fluorescent primer target sequence. In an embodiment, the 3′ end of the probes are phosphorylated or include an inverted dT or Dideoxycytidine (ddC) to block any potential unwanted PCR extension of the probes. Customized DNA probes can be purchased from vendors, such as Integrated DNA Technologies, that are already modified with an acrydite group to facilitate direct incorporation of the probes into particles during hydrogel polymerization, as is described below.

There is a melting temperature associated with every pair of nucleic acid oligonucleotides in a given buffer. Common factors that increase the melting temperature are (1) a high degree of complementarity, (2) high salt concentrations, and (3) a high proportion of guanine and cytosine nucleotides. As temperature is increased near and beyond the Tm of a pair of oligonucleotides, hybridized complexes become less stable, and therefore de-hybridization will occur. This principle determines the specificity of hybridization, implying that hybridization should be carried out above the Tm of any undesirable oligonucleotide complexes but below that of the desired complexes to avoid loss of signal. In the present PCR methods, signal and specificity are optimized by selecting a hybridization temperature that is at least about 2° C. to about 15° C. higher than the selected primer annealing temperature, but lower than the Tm of target-probe complexes. Typically the primer annealing temperature is from about 35° C. to about 60° C. and the hybridization temperature is from about 37 to about 75° C. This is accomplished by designing primers and probes that have melting temperatures relatively far apart to minimize unwanted amplicon/probe hybridization. In a preferred embodiment a primer is designed that has a Tₘ of about 55° C., probes are designed that have a Tₘ above about 70° C., primer annealing is conducted at a primer annealing temperature in the range of about 52-55° C., and the hybridization step is conducted at a hybridization temperature of about 63-70° C., which is between the primer Tₘ and the Tₘ of the probe-amplicon complex. Alternatively, probes can be modified with locked nucleic acids to increase the Tm of probe-amplicon complexes, even beyond the amplicon Tm. This is particularly attractive as hybridizations may be performed at temperatures above the temperature at which amplicons remain hybridized to their complementary oligomers in solution. This ensures that amplicons will preferentially bind to the particles, increasing signal for detection. The length of the hybridization time affects the amount of target bound to the particles. In an embodiment using porous hydrogel particles, hybridization times typically range from about 10 minutes to an hour or more; longer times are preferred to maximize sensitivity. (Pregibon et al., Anal. Chem. 2009, 81, 4873-4881.)

Samples of nucleic acid targets are prepared using standard procedures including biochemical purification, enrichment, immunological or physical treatments (PCR Technologies: Current Innovations, Thomas Weissensteiner, Hugh G. Griffin, Annette M. Griffin). There are also several commercial kits available for PCR sample preparation.

### One-step Quantitative Multiplexed End Point PCR

Polymerase chain reaction (PCR) is a technique widely used in molecular biology to amplify a piece of DNA by in vitro enzymatic replication. Typically a technician must intervene between amplification and hybridization cycles, raising the risk of contamination and making the process relatively slow at about 8 hours per experiment. Others have recently described a one chamber multiplexed real time PCR assay using probes immobilized on particles; however this method requires that the steps of performing a series of amplification cycles followed by hybridizing the labeled amplification products to probes be repeated at least twice to quantify the amount of target in the sample. Whitman et al. US Application Serial No. 2008/0305481.

By contrast, embodiments of the methods of the present invention permit quantification of the amount of a particular nucleic acid target in a complex sample using a one chamber multiplexed quantitative end point PCR particle assay that has only one set of amplification cycles followed by a single hybridization step. In a preferred embodiment the particles are encoded hydrogel particles with more than one probe region permitting quantification of more than one target on a single particle and the amplicons are fluorescently labeled. The signal from the labeled amplicons bound to the probes is detected and the amount of fluorescence emitted from each probe region on the particle is quantified, preferably by passing the particles one at a time through a flow cytometer equipped for fluorescence detection. The amount of nucleic acid target in the complex sample is determined by comparing the quantified amplicon signal to either 1) a standard curve that correlates the signal generated by binding of a nucleic acid target to the corresponding probe over a broad range of target concentrations, 2) an endogenous reference gene or 3) by adding a known amount of an external target nucleic acid to the sample. Methods for quantifying the amount of a nucleic acid target in a sample after PCR by comparison to an amplified endogenous reference gene such as beta-actin, GAPDH, nucleolar RNA or 18S ribosomal RNA, are described in I. Nezarenko, et al, Nucleic Acids Research 2002, Vol. 30, No. 9 e37. The results of the quantitative PCR using fluorogenic primers can be analyzed by the comparative C_{T} method (User Bulletin 2, ABI PRISM 7700 Sequence Detection System, P/N 4303859). This method of analysis does not require plotting of a standard curve of C_{T} versus starting copy number. Instead, the amount of target is calculated based on the difference between the C_{T} of the target and an endogenous reference gene. Example 3 describes quantifying the amount of target nucleic acids by comparison to an endogenous reference gene.

In a preferred embodiment, hydrogel particles are analyzed with high-throughput particle scanning in flow-focusing microfluidic devices that read the codes and determine the amount signal from the fluorescently labeled amplicons bound to the appropriate respective complementary probe region. Pregibon, D. C., M. Toner, and P. S. Doyle, Multifunctional encoded particles for high-throughput biomolecule analysis. Science, 2007. 315(5817): p. 1393-1396. The devices are preferably scanned using slit illumination where the fluorescence intensity is measured using a photomultiplier module (such as Hamamatsu H7422™) and particle code and target signal are decoded in real-time using custom written scripts.

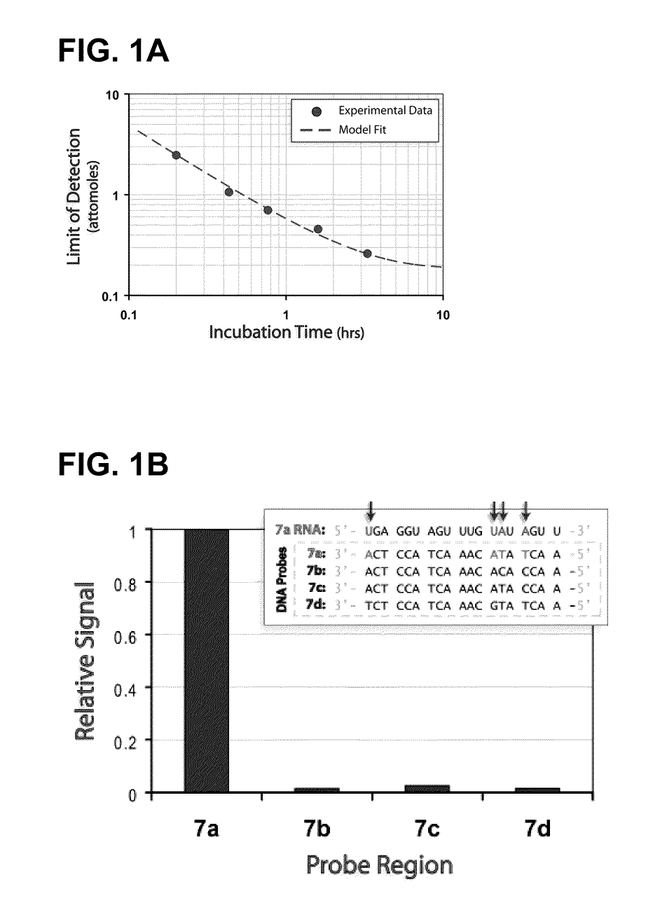

Proof of concept experiments are described in detail in the Examples. Most of the experiments described herein utilized encoded hydrogel particles. An important metric for nucleic acid detection is specificity, e.g. how well the assay can distinguish between closely-related targets. In order to show that the hydrogel particle design and labeling scheme did not negatively affect specificity, cross-reactivity of an RNA target with closely-related complementary probes was determined. Four different microRNAs from the let-7 family that varied by only one or two nucleotides in sequence were amplified and detected using multiplexed end point PCR. Example 1, FIG. 1. The results showed that encoded hydrogel particles provided single-nucleotide specificity with less than 3% cross-reactivity with sub-attomole sensitivity even without using probes with locked nucleic acids. This level of sensitivity far exceeds the ˜100 attomol sensitivity reported for the current state-of-the art bead-based system.

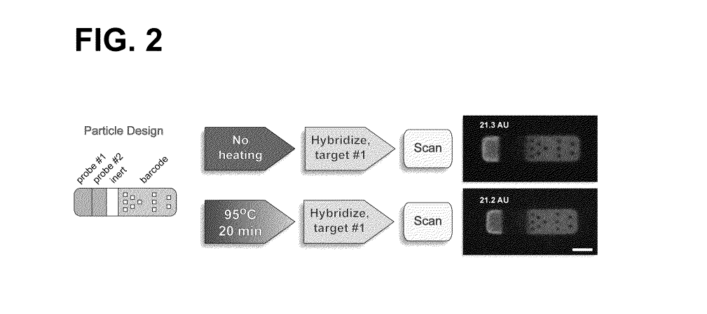

Further testing of encoded hydrogel particles showed that they were heat-stable under conditions used for PCR without deforming or loosing sensitivity. Example 1, FIG. 2.

One chamber multiplexed end point-PCR amplification and detection of two unique targets on λ-phage DNA was conducted using selected target sequences that were 60 base pairs long and had no homology to homo sapiens in a BLAST search. The sense strand of the λ-phage DNA was the target for detection, therefore the reverse primer was designed to incorporate a 5′ Cy3 modification. Asymmetric primer concentrations were used. Forward and reverse PCR primers (SEQ ID NOS: 1 and 2, respectively) twenty base pairs long were designed with a melting temperature Tₘ near 55° C. and minimal hairpin formation or interaction. The reverse primer did not have sufficient complementary to the probe to permit nonspecific binding. Probe sequences SEQ ID NOS: 3 and 4 were designed that (1) were complementary to a 40 base pair portion of the target sequence, (2) had a melting temperature Tₘ above 72° C., and (3) were unable to form stable hybrids with the labeled primer due to a lack of complementarity. The probe length of 40 base pairs was selected so that it formed a stable hybrid molecule with the amplicon at the 63° C. hybridization temperature. Each probe was incorporated into the appropriate probe region of a barcoded hydrogel particle. Details of the experiment are set forth in Example 2.

A hybridization temperature of 63° C. that was 14° C. higher than the annealing temperature was used. As described above, the hybridization temperature is preferably 2-15° C. higher than the annealing temperature to maximize sensitivity and specificity. After one hour hybridization, the sample (that now includes labeled amplicons #1 and #2 hybridized to respective probes #1 and #2 at known sites on the encoded hydrogel particles) was diluted and each particle was directly imaged using a fluorescence microscope and EB-CCD camera to quantify the amount of fluorescent signal from each labeled amplicon. There is no requirement for washing the particles prior to scanning with the present methods.

To ensure a strong signal for the lambda phage experiments, a higher than necessary amount of target nucleic acids were used in these proof of concept experiments; however, hydrogel particles are sensitive to sub-attomole amounts of targeted nucleic acids as described above. Hybridization times can vary. Because porous hydrogel particles have probe molecules dispersed throughout the matrix, a longer hybridization time of one hour were used to optimize diffusion of the labeled amplicons into the hydrogel. Hybridization times for hydrogel particles are preferably from about 10 minutes to about one hour. Other types of particles where probes are immobilized only on the outer surface typically require a shorter hybridization times. Routine experimentation will determine the optimum hybridization time and hybridization temperature depending on the particles, primers and probes that are used.

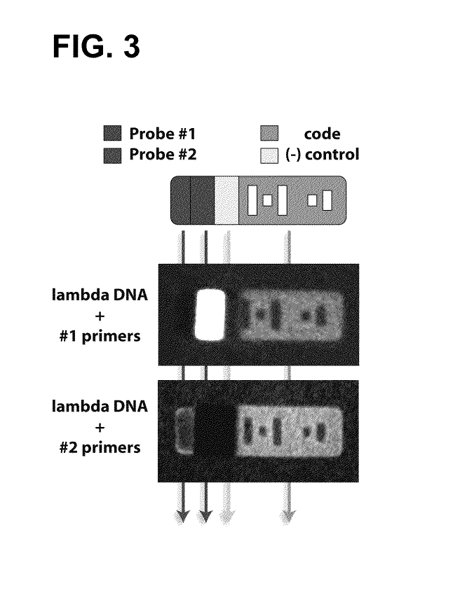

These experiments using individual Cy3-labeled primers for target #1 or target #2 λ-phage DNA showed that amplification and detection of the intended DNA targets using the new one pot multiplexed end point PCR method with a hybridization temperature that was significantly higher (14° C.) than the primer annealing temperature was both highly sensitive and specific. Although amplification of target 1 appeared to be much more efficient than that of target 2, the efficiency of amplification of target 2 can be increased with routine experimentation by optimizing the design of probe #2, optimizing primer concentrations, or using more PCR cycles. FIG. 3. The amount of fluorescence from each particle region was quantitated, and could be compared with a standard curve to quantitate the amount of the respective nucleic acid target in the original sample as is described below. A qualitative positive result required probe-region fluorescence to be about 10× above the background noise.

By quantifying the amount of target nucleic acid in the sample is meant both calculating the actual amount (the absolute number of copies of the target before amplification) and the relative amount of the target (normalized either to a known amount of exogenous DNA input or to endogenous reference genes/targets also called normalizing genes/targets). The amount of a target amplified over multiple amplification cycles reaches a plateau after for example about 30 cycles and the amount is strongly affected by the amount of primers used.

The actual amounts of RNA or DNA can be approximated by comparing the results to a standard curve produced by PCR using serial dilutions (eg. 1, 10, 100, 1000 copies) of a known amount of RNA or DNA. In another embodiment, the amount of a nucleic acid target in different samples is determined using a reference gene and dividing the measured amount of fluorescence from the labeled amplification products by the amount of fluorescence from a reference gene present in the same sample to normalize for possible variation between different samples. A reference gene is selected that is expressed equally across all the samples. Commonly used reference genes are the endogenous genes for beta-actin, GADPH, 18S RNA and nucleolar RNA. Nailis H, et al. (2006), “Development and evaluation of different normalization strategies for gene expression studies in Candida albicans biofilms by real-time PCR”. BMC Mol Biol. 7: 25. and Nolan T, et al., (2006). “Quantification of mRNA using real-time RT-PCR.”. Nat. Protoc. 1: 1559-1582.

Gene expression profiling experiment were conducted to detect and quantify the effects of drug treatment on expression of certain target mRNAs in a complex sample using hydrogel particles. Applicants repeated previously reported multiplex ligation-mediated PCR experiments that used Luminex FlexMAP (Luminex, Austin, Tex., USA) optically addressed barcoded microspheres in side by side experiments with hydrogel particles using an endogenous reference gene for quantification. The methods used are described in Peck et al. A Method for High-Throughput Gene Expression Signature Analysis, Genome Biology, 7:R61, 2006; details are set forth in Example 3. Although the Peck et al experiments were done with traditional RT-PCR and hybridization in multiple steps, the experiments are amendable to embodiments of the present methods for one chamber quantitative multiplexed end point PCR.

HL60 (human promyelocytic leukemia) cells were cultured in the presence of either 0.1% tretinoin in DMSO or DMSO alone, and quantification was based on comparison of target mRNA expression to the reference gene GADPH3. Four mRNAs that Peck showed were responsive to drug treatment (designated LUA68, LUA59, LUA27, and LUA7) and the same internal reference target GADPH3 (designated LUA95) were selected for amplification. Upstream and downstream probe pairs designed for each of the targets by Peck were used to generate the amplicons. Unique upstream biotin-conjugated primers for each target included a 24 nucleotide bar code sequence for each of the five mRNAs. Applicants designed probes for immobilization on encoded hydrogel particles that were complementary to the 24 nucleotide bar code on each unique upstream primer to enable capture of the amplicons on encoded hydrogel particles. Methods for making the particles are described in Example 3. Each encoded hydrogel particle was made with multiple probe regions directed to each of the five mRNAs (four targets and one reference gene) and a control, respectively.

Total RNA was isolated from the HL60 cells, reverse-transcribed and amplified using a simple RT-PCR method. In order to compare the sensitivity of the encoded hydrogel particles to the Luminex magnetic particles, the exact protocol described by Peck et al. was followed. There were 39 cycles in which: there was an initial denaturation at 92° C. for 9 minutes, followed by denaturation at 92° C. for 30 s, followed by annealing at 60° C. for 30 s, extension at 72° C. for 30 s for 39 cycles, and a final extension at 72° C. for 5 minutes. After a final denaturation at 95° C. for two minutes hybridization was carried out at 45° C. for 60 minutes. Note that in the preferred embodiments of the present invention the hybridization temperature is from 2-15° C. higher than the primer annealing temperature to optimize specificity. By contrast, the hybridization temperature (45° C.) used by Peck et al. was 15° C. lower than the annealing temperature.

After hybridization, the respective Luminex and hydrogel particles were rinsed, incubated with streptavidin-phycoerythrin to fluorescently label the primers, rinsed again, and imaged for fluorescence. The fluorescent signals obtained were quantified and normalized against the internal reference (LUA95) after subtracting the background signal from the control region on each particle (CTL).

The results showed that encoded hydrogel particles on which complementary probes to the five targets were immobilized accurately captured the up- and down-regulation of mRNA targets with tretinoin treatment. Quantification of target expression against the reference gene showed that the hydrogel particles showed quantitatively similar expression profiles (Table 1).

The relative amount of fluorescence compared to the internal reference target after hybridization indicates the quantity of nucleic acid target (template) that was in the original sample. Note that for qualitative PCR, it is ideal to completely deplete the labeled primers to get the brightest signal possible. By contrast, for quantitative multiplexed end point PCR, it is not desirable to deplete the primers because the level of the signal is variable and depends on the number of target templates initially present in the sample.

TABLE 1

| DMSO | | tretinoin | | Differential |
|---|---|---|---|---|
| Raw | Normalized | Raw | Normalized | Expression: |
| Signal (AU) | Signal | Signal (AU) | Signal | log(tret/DMSO) |

Encoded Hydrogel Particles

| | | | | | |
|---|---|---|---|---|---|
| LUA95 | 200.16 | | 88.45 | | |
| LUA68 | 44.98 | 0.225 | 2.99 | 0.034 | −0.822 |
| LUA59 | 17.95 | 0.090 | 1.14 | 0.013 | −0.841 |
| LUA27 | 5.04 | 0.025 | 14.64 | 0.166 | 0.818 |
| LUA7 | 5.50 | 0.027 | 7.44 | 0.084 | 0.486 |

Luminex

| | | | | | |
|---|---|---|---|---|---|
| LUA95 | 2789.1 | | 1754.4 | | |
| LUA68 | 1177.8 | 0.422 | 178.0 | 0.101 | −0.619 |
| LUA59 | 600.5 | 0.215 | 100.3 | 0.057 | −0.576 |
| LUA27 | 165.8 | 0.059 | 775.5 | 0.442 | 0.871 |
| LUA7 | 101.5 | 0.036 | 389.3 | 0.222 | 0.785 |

Table 1: Raw and normalized data for a gene expression profiling experiment. For encoded hydrogel particles, signal calculated from five particles were averaged. The negative control signal [(−) CTL] was subtracted from each probe-region signal. These signals were then normalized using the internal reference (LUA95) to find the relative expression of each target in the samples. These expressions were compared for drug-treated and non-treated samples (by calculating the ratio). Differential expression is found by taking the logarithm of this expression ratio.

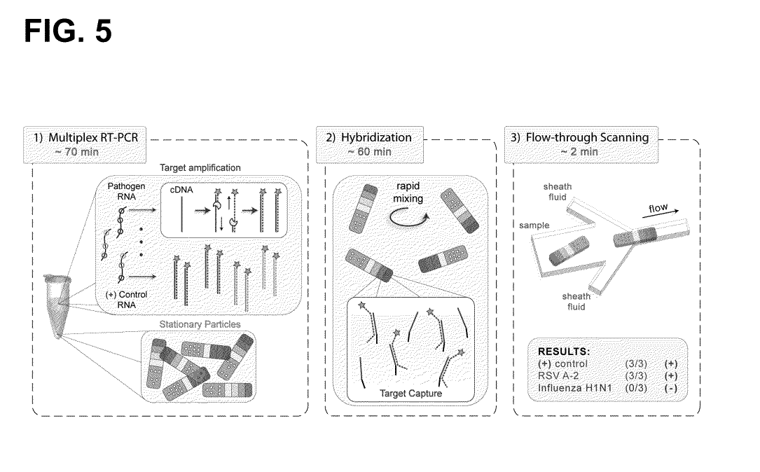

The new methods of the present invention are also suitable for detecting the presence of any pathogen, including DNA or RNA viruses, bacteria, and fungi, in a biological or environmental sample. Example 3 provides a detailed outline for setting up and optimizing an assay for detecting and quantifying an influenza RNA virus (such as influenza a (h1N1) and respiratory syncytial virus (RSV) (such as A-2) using commonly-targeted, highly-conserved genomic regions of the respective viruses as nucleic acid targets. FIG. 5. Persons of skill in the art can adapt these methods for setting up assays for other pathogens such as those causing sexually transmitted diseases (STDs). Using the present multiplexed end point PCR amplification and quantification methods, multiple STDs such as N. gonorrhoeae and C. trachomatis can be screened in a single specimen.

### SNP Detection Using Multiplexed End Point PCR.

Another embodiment of the present invention is directed to a two-color method for detecting genetic variations including single nucleotide polymorphisms (SNPs) in a complex sample, which will enable the diagnosis and screening of many different genetic disorders. This will broadly benefit patients and carriers of undiagnosed genetic diseases and enabling high-throughput association studies for disorders not previously investigated due to cost prohibitive technology. The present methods can be adapted as the genetic origins of more diseases are elucidated.

The genetic origins of these disorders may be monogenic as seen with disorders like cystic fibrosis, or may be extremely complex involving the interaction of several genes, as is the case with many cancers. More than 4,000 specific gene variants have been associated with common diseases such as heart disease, diabetes, asthma, and cancer. Rare diseases are those that occur at frequencies less than 1 in 2,000 people in a population, suggesting that these disorders affect only a small number of people. However, it is estimated that there are up to 8,000 rare diseases, cumulatively affecting up to 8% of the total population.

Genetic disorders can be caused by a number of DNA mutations including insertions, deletions, point mutations, single nucleotide polymorphisms (SNPs), and more. However, of the genetic variations observed in the human genome, nearly 90% are accounted for by SNPs. Collins, F. S., L. D. Brooks, and A. Chakravarti, A DNA polymorphism discovery resource for research on human genetic variation. Genome Res, 1998. 8(12): p. 1229-31.

Two technologies that have been used frequently to successfully identify SNPs are sequencing and real-time PCR. Unfortunately, these approaches are both expensive, provide a very low throughput, and are not particularly amenable to implementation in clinical settings. Microarrays have been used for these and other high-density assays, but are known to be expensive, not reproducible, and low-throughput.

The results described above showed that hydrogel encoded particles provide sub-attomole sensitivity and single-nucleotide specificity with less than 3% cross-reactivity, even without the use of locked nucleic acids or altered probe design (FIG. 1), which makes them ideal for SNP detection. Two methods for detecting single nucleotide mutations are known: (1) one uses allele-specific probes for selective target capture, and (2) one uses allele-specific primers for selective labeling of target genes. An allele is one of a series of different forms of a gene, i.e. wild type and mutant forms.

### Allele-Specific Probes for SNP Detection

One chamber multiplexed end point PCR can be used for the detection of genetic variation (including SNPs) using allele-specific probes that selectively hybridize to either the wild type or one of the mutant alleles of a gene of interest. Ordinary primers can be used with allele-specific probes as described above. In certain other embodiments, the allele specific probes have one or multiple locked nucleic acid (LNA) modifications in order to increase stability of properly hybridized alleles. Probe Tm can cover a wide range, but certain embodiments the probe Tm for SNP detection are near 55° C. (or 57-59° C. for LNA-modified probes) For SNP detection, need to keep probes short to get best discrimination, and they are capped, for example by 3′ phosphorylation, to avoid incidental extension during PCR.

In a preferred embodiment, amplicons for SNP detection are from about 50 to about 70 base pairs in length, primers are preferably ˜20 base pairs in length, and DNA probes are approximately 20 base pairs long (or shorter). Separate probes are designed to target the interior region of each allele, such that the mutation site is approximately centered in the probe sequence. In an embodiment the probe is 3′ phosphorylated or otherwise capped, for example using inverted dT or ddC, to avoid incidental extension during PCR. In this embodiment, scanning is done using a single color and multiple probe regions.

Although the embodiments of the present methods using encoded hydrogel particle hybridizations are highly specific and discriminate single-nucleotide differences even using un-modified DNA probes and primers, one embodiment for SNP detection incorporates a single LNA-modified nucleotide at the SNP point mutation site on the allele-specific primers (for both the wild type or mutant primers) to raise the Tₘ of a perfect primer/target match by 2-4° C. in order to optimize assay specificity. This creates a separation of melting temperatures for match and mismatched pairs making it easier to discriminate between the two. Castoldi, M., et al., RNA-a Publication of the RNA Society, 2006. 12(5): p. 913-920) In the allele-specific primer amplification method, the primers would have the LNA. Only the appropriate allele will be amplified. In the other method using allele-specific probes, both alleles are amplified and an LNA is used on the probe to preferentially hybridize to only one of those.

### Allele-Specific Primers for SNP Detection

Applicants also describe a method for detecting genetic variation (including SNPs) herein referred to as a “multiple-color genetic variation detection method,” using one chamber multiplexed endpoint PCR and differentially labeled allele-specific primers. An embodiment is directed to detection of genetic variation using different allele-specific primers that are differentially labeled with two or more distinct reporters such as two unique fluorophores (for example, Cy3 for a wild type allele and Cy5 for a mutant allele), and a single probe that can bind to either the wild type or mutant amplicon. The primers could also be labeled with different radioisotopes or quantum dots, for example. The probe is designed to be complementary to a region of the targeted gene that is adjacent to (flanks) or slightly overlapping the mutation site and therefore is common to both of the alleles being tested, for example a mutant and a wild-type allele. As such, each probe can bind either wild-type or mutant amplicons. A comparison of the relative intensities of the two fluorophores determines which allele(s) is/are present in the sample. A sample from a patient, for example, would be screened to determine whether the patient carries the wild type allele or one of possibly many different mutant alleles. In other embodiments, up to six different alleles labeled respectively with six unique reporters can be used in this method. A person of skill in the art can vary the method to optimize the number of different alleles, for example using both differentially labeled primers that could include fluorophores, quantum dots, radioisotopes, etc.

Certain embodiments are directed to a method of amplifying and detecting genetic variation at a known mutation site in a gene in a sample comprising a plurality of nucleic acid targets by:
(a) combining in a chamber
   a first primer pair for priming amplification of a first allele of the gene using multiplex PCR, wherein the primers hybridize to the targets at a primer annealing temperature, and wherein one primer of the first primer pair is labeled with a first reporter, and
   a second primer pair for priming amplification of a second allele of the gene using multiplex PCR, wherein the primers hybridize to the targets at a primer annealing temperature, and wherein one primer of the first primer pair is labeled with a second reporter,
   a plurality of particles on which are immobilized a plurality of nucleic acid probes that are complementary to a nucleic acid sequence that is common to both the first and second alleles, wherein the common sequence is adjacent to the known mutation site, and a PCR cocktail containing enzymes for amplifying nucleic acid targets,
(b) performing one or more amplification cycles to form labeled amplification products for the first and second alleles,
(c) hybridizing the labeled amplification products to the probes at a hybridization temperature that is at least about 2° C. to 15° C. degrees higher than the primer annealing temperature, and
(d) detecting a signal from the labeled amplification products hybridized to the probes on the particles at a first wave length corresponding to the first reporter and at a second wave length corresponding to the second reporter, and comparing the two signals thereby detecting the relative quantities of the first and the second alleles on the particle.

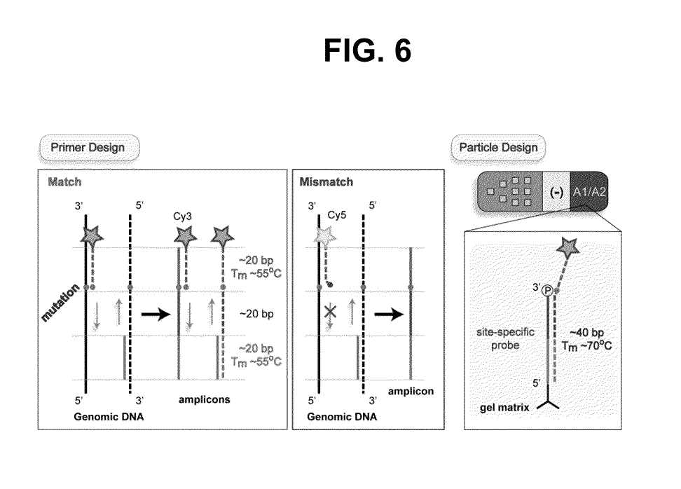

It is well known that primers with mismatches near their 3′ end are not suitable for PCR. In order to exploit this fact, the present embodiments use allele-specific primers wherein the site of genetic variability is present at the. 3′ end of each allele-specific primer to assure accurate hybridization, i.e. the nucleotide residing at the mutation site in the gene of interest (either the wild type or mutant allele) is located at the 3′ end of the primer. Unless there is 100% complementarity to the target, the primer will not be extended. Thus, the primer for the mutant allele will only be extended if the targeted gene includes the particular mutation at the mutation site. If the patient has the mutant allele, the primer for the wild-type allele will not be able to form a stable hybrid with the gene. FIG. 6.

In preferred embodiments of genetic variation analysis using the present methods, primer design and assay conditions are optimized to ensure specific and accurate identification of all targets from heterozygous and homozygous alleles. For any mutations that are extremely rare and for which genomic DNA is not commercially available, synthetic DNA can be used during optimization experiments in order to ensure that mutation and wild-type alleles are accurately discriminated.

Among genetic diseases that can be diagnosed using the present PCR dual fluorescence SNP detection method for is cystic fibrosis, the most common inherited autosomal recessive disorder, with a carrier frequency of 1 in 25 in the Caucasian population. This life threatening disease affects the respiratory, digestive, exocrine, and reproductive systems. While there are over 1000 known mutations that can lead to disease, the American College of Medical Genetics (ACMG) has recommended that 23 specific SNP mutations of the CF transmembrane regulator gene (CFTR) be including in CF screening Table 1. Watson, M. S., et al., Cystic fibrosis population carrier screening: 2004 revision of American College of Medical Genetics mutation panel. Genet Med, 2004. 6(5): p. 387-91. The detection of all 23 CFTR mutations with 100% specificity in a single sample is possible using the present one chamber, multiplexed dual color PCR assay for SNP detection. In such an assay there would be allele-specific primers for each of the different SNPs with a primer for the corresponding wild type allele. Using coded particles, a single sample can be assayed for multiple SNPs. The assay can be validated using genomic DNA from cystic fibrosis patients, carriers, and negative controls.

Genomic DNA can be obtained from patients, carriers, and negative which will provide both homo- and heterozygous alleles. For assays, approximately 1,000 copies of DNA template could be used in 50 μl PCR samples with about 30-40 amplification cycles for qualitative analysis using excess labeled primer.

### Optimization of Assay Conditions

Assay conditions can be optimized by persons of skill in the art depending on the particular experiment. It is not uncommon for PCR reactions to be nearly 100% efficient, meaning that product is doubled every single amplification cycle. Thus, for every molecule of starting template, there will be approximately 10⁶ (1.7 attomol), 10⁹ (1.8 fmol), and 10¹² (1.8 pmol) product molecules after 20, 30, and 40 cycles, respectively. Serial dilutions of known target DNA or RNA ranging from 10⁴ to ˜1 can be used to determine the optimum amount of template for confident detection in a given assay. Run-to-run variability can be determined by repeating each test multiple times. Detection of <10 copies of target DNA or RNA with an assay that requires less than 2 hours total is ideal and is possible with the new methods.

For accurate quantification of amplicon binding using a one-pot assay with fluorescently-labeled primers, the concentration of fluorescent signal from the targets bound to the probes must be much greater than the background fluorescence from unreacted labeled primers in the solution. Using hydrogel particles and a one-hour hybridization after PCR amplification, the signal obtained from target capture is calculated to be sufficiently high to perform a 240-plex assay with signal-to-background ratio of 100, as found using the models developed in (Pregibon, et al., Anal. Chem. 2009, 81, 4873-4881) to estimate bound targets.

The presence of particles in the PCR reaction has no effect on the amplification reaction. While the particles are confined to a small volume in the bottom of the reaction tube, the PCR reactants/templates are evenly distributed throughout the tube (due to Brownian motion). Further, the discrepancy between PCR volume to particle volume (>1000:1) ensures that the PCR reaction dominates over hybridization of product to the particles. Any PCR product captured by the particles during an annealing step was released in the next cycle during the high-temp denaturing step. In a preferred embodiment, probes for use in the present methods do not include primer-complementary sequences that would allow the probes to form stable hybrids with the primers. Further, while the relative timescale for hybridization to particles for low target concentrations is typically ˜1 hour, each PCR amplification cycle lasts only ˜45 seconds −5 minutes. This discrepancy in timescale further minimizes interference of the particles on PCR amplification. The amount of free PCR primers diminishes over the amplification cycles by the time the hybridization step begins. Hybridization is preferably accompanied by mixing to facilitate continuous sampling of the reaction mixture by the particles, though mixing is not required. In most cases, an excess of the primer that is labeled is used to ensure that there are target-strand amplicons free for hybridization. However, in the embodiments where probes are modified with locked nucleic acids to provide probe-target melting temperatures above the amplicon melting temperatures, this biasing is not necessary.

In a typical assay, about 30 particles per target nucleic acid are used (3 μl of particles at 10 particles/μl) are pipetted into the chamber with them PCR product for a total sample volume of about 50 microliters in a 0.65 ml Eppendorf tube. In an embodiment, hybridization is carried out at a temperature of about 60° C. with an annealing temperature of about 55° C. for one hour with rapid mixing typically at 1800 rpm in a thermomixer (Rio, Quantafoil). For hydrogel particles, a one hour hybridization after the PCR amplification generated a signal obtained from target capture that was sufficiently high to perform a 240-plex assay with a signal to background ratio of about 100. The hybridization times will vary depending on the particles used.

The present invention can also be used qualitatively. The amount of primers used affects (1) the amount of product made, (2) the ratio of sense (detected) to anti-sense amplicons, and (3) the level of background fluorescence during scanning A model system to investigate the use of primers at varying concentrations and ratios can be used to optimize a particular assay to qualitatively access the presence of one or more nucleic acid targets in a sample. Table 2 provides the design of a PCR primer concentration study with varying base concentrations and ratios for forward/reverse primers. In this study amplicons incorporating the forward primer labeled with Cy3 can be used subsequently for hybridization and detection. All concentrations should be orders of magnitude above the detection limit of the scanning system.

TABLE 2

| Trial | Forward (detected) | Reverse | Ratio |
|---|---|---|---|
| 1 | 500 attomol (10 pM) | 500 attomol (10 pM) | 1:1 |
| 2 | 100 attomol (10 pM) | 50 attomol (10 pM) | 2:1 |
| 3 | 1 fmol (20 pM) | 500 attomol (10 pM) | 2:1 |
| 4 | 10 fmol (40 pM) | 5 fmol (20 pM) | 2:1 |
| 5 | 2 fmol (40 pM) | 500 attomol (10 pM) | 4:1 |

Additional reagents known to those skilled in the art may be used to improve the efficiency of any of the embodiments described herein. BSA or other molecules which act as blocking agents or detergents or surfactants may also improve the methods described herein. BSA or other similar reagents known to those skilled in the art may improve a PCR reaction performed in a glass or quartz chamber by reducing the attraction of DNA and other reagents to the surfaces of the reaction chamber. Other additives may be used which exhibit molecular crowding effects to improve the hybridization time, such as nucleic acid binding proteins or minor groove binders.

### Particle Design and Preparation

The poly(ethylene glycol) hydrogels, a preferred embodiment for encoded particles, provide solution-like thermodynamics (i.e. strong binding) that allows high sensitivity and specificity, and three-dimensional probe distribution that gives a high target capacity to accommodate a wide dynamic range. The particles are robust, withstanding high-temperatures used in PCR.

Hydrogels are a class of bio-friendly materials that characteristically retain water, allowing biological interactions to occur in three-dimensional space. Hydrogel materials (e.g. poly(ethylene glycol), PEG) are non-fouling, thus limiting non-specific interactions, and can derive from an extremely broad list of precursors. Nucleic acid hybridization in gels closely resembles that in solution, which enhances the sensitivity and specificity of nucleic acid detection. Fotin, A. V., et al., Parallel thermodynamic analysis of duplexes on oligodeoxyribonucleotide microchips. Nucleic Acids Research, 1998. 26(6): p. 1515-1521.

Hydrogel particles are synthesized using “flow lithography” that combines the non-mixing and continuous nature of microfluidics with the precise sculpting of photolithography to control microparticle morphology, chemistry, and functionality. Pregibon, D. C., M. Toner, and P. S. Doyle, Multifunctional encoded particles for high-throughput biomolecule analysis. Science, 2007. 315(5817): p. 1393-1396; Dendukuri, D., et al., Stop-flow lithography in a microfluidic device. Lab on a Chip, 2007. 7(7): p. 818-828; Dendukuri, D., T. A. Hatton, and P. S. Doyle, Synthesis and self-assembly of amphiphilic polymeric microparticles. Langmuir, 2007. 23(8): p. 4669-4674; Dendukuri, D., et al., Continuous-flow lithography for high-throughput microparticle synthesis. Nature Materials, 2006. 5(5): p. 365-369; and Panda, P., et al., Stop-flow lithography to generate cell-laden microgel particles. Lab on a Chip, 2008. 8(7): p. 1056-1061; and US Patent Applications 2007/0105972 and 2008/0176216. Flow lithography is high-throughput (10⁸ particles/hr), high resolution (˜1 □m), can form particles with multiple adjacent functionalities, and is applicable to any precursor material that reacts via free-radical polymerization.

In an embodiment hydrogel particles contain blends of PEG-diacrylate (n=700, Sigma), and inert PEG (n=200, Sigma) to tune pore size of the resulting hydrogel. In certain embodiments probe regions contain 20% PEG-diacrylate and 40% PEG, while encoded regions contain 30% of each. The encoded region contains a fluorescent dye, such as rhodamine-methacrylate (Polysciences) for visualization. In other embodiments, particles are used that have a greatest particle dimension less than about 500 micrometers (μm, 1 μm=10⁻⁶ meters), and an aspect ratio of length to width greater than about three, wherein the encoded portion is more rigid and less porous than the rest of the particle, including the probe portion. This is an advantage because the encoded portion better retains its structure to reduce errors in reading the code, while the increased porosity of the remaining portion allows rapid diffusion of targets into the hydrogel matrix for a better signal strength related to binding. In an embodiment the more rigid hydrogel composition includes about 30% Poly(ethylene glycol) (700) diacrylate and about 30% Poly(ethylene glycol) (200), called DA30 hereinafter, where 700 and 200 refer to the molecular weights of the corresponding polymers; and the more porous hydrogel composition includes about 20% Poly(ethylene glycol) (700) diacrylate and about 40% Poly(ethylene glycol) (200), called DA20 hereinafter. (Chapin, S., Pregibon, D. C., and Doyle, P. S., “High-throughput flow alignment of barcoded hydrogel microparticles”, Lab Chip, 9, 3100-3109, 2009.)

### Kits

The present invention also provides kits containing components for use in various embodiments of the multiplexed end point quantitative PCR assays. Any of the components disclosed herein may be combined in a kit. In certain embodiments the kits include one or more primer pairs for priming amplification of one or more corresponding nucleic acid targets, preferably pre-labeled with fluorescent labels, and one or more particles on which are immobilized a plurality of probes complementary to a corresponding nucleic acid targets, preferably encoded hydrogel particles. In certain embodiments the particles are encoded polymer particles with multiple probe regions. The kit may include probes that the user can complex with a desired particle. DNA polymerase and/or reverse transcriptase can also be included.

In an embodiment of a kit for detection of genetic variants such as SNP detection, the kit includes allele-specific pre-labeled primers as described above for -color detection using up to about six differentially labeled primers, wherein one primer is specific for a first allele such as a wild type allele, and the others are specific for other alleles showing the genetic variations, such as a mutant allele comprising the SNP. The kit further includes probes that will bind to any of the allele-specific primers for the gene, which probes can be either free or more preferably immobilized onto particles, preferably hydrogel particles.

Because the assay is multiplexed, the kit can include a plurality of different particles each having a different probe or set of probes immobilized thereon, and a plurality of different primer pairs. If the primers are not pre-labeled, the kit optionally includes a detectable label that specifically binds to the primer. Two probe kits come within the scope of the invention also, in which one probe is labeled or is modified to bind to a labeling agent.

The components of the kits may be packaged either in aqueous media or in lyophilized form. The container means of the kits will generally include at least one container, into which a component may be placed, and preferably, suitably aliquoted. An appropriate number of containers will be included to accommodate each of the components in the kit. In some embodiments more than one component may be included in a single container. The kits of the present invention also will typically include packaging for containing the various containers in close confinement for commercial sale.

When the components of the kit are provided in one or more liquid solutions, the liquid solution may be an aqueous solution, with a sterile aqueous solution being particularly preferred. However, certain components of the kit may be provided as dried powder(s). In certain embodiments the hydrogel particles may be dried. When reagents and/or components are provided as a dry powder, the powder can be reconstituted by the addition of a suitable solvent that may be included in the kit or provided separately. A kit may also include instructions for employing the kit components. Instructions may include variations that can be implemented.

### Nucleic Acids

In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Hybridization temperature as used herein specifically refers to the temperature at which amplicons hybridize to complementary probes.

“Complementary,” as used herein, refers to the capacity for precise pairing between two polynucleotides. For example, if a nucleotide at a certain position of a nucleic acid is capable of hydrogen bonding with a nucleotide at the same position of a DNA or RNA molecule, then the nucleic acid and the DNA or RNA are considered to be complementary to each other at that position. Perfect complementarity is not required throughout the length of the primers and probes. In the allele-specific primers, for example, the goal is to capture “similar” targets on the same probe, like the point mutated alleles that have been labeled with different fluorophores. There also might be instances where it is desirable to capture all targets from variable regions of genes when the exact sequences of the variable segments are not known. For primers, 100% complementarity is not necessary along the entire primer (and actually leads to “non-specific amplificaiton”), though it is very important to have complementarity for the few base pairs near the 3′ end of the primer (where extension occurs). A mismatch at the 3′ end would dramatically inhibit if not block primer extension. A mismatch at the 5′ end will have little effect. Typically, one would want to have perfect matching of probe/target, though this is not necessary. Persons of skill in the art are aware of complementarity issues for PCR.

Two nucleic acids are complementary to each other when a sufficient number of corresponding positions in each molecule are occupied by nucleotides which can hydrogen bond with each other. Thus, “complementary” indicate a sufficient degree of complementarity or precise pairing such that stable and specific binding occurs between the two nucleic acids. As used herein “stringent conditions” or “high stringency” are those conditions that allow hybridization between or within one or more nucleic acid strands containing complementary sequences, but preclude hybridization of non-complementary sequences. Such conditions are well known to those of ordinary skill in the art, and are preferred for applications requiring high selectivity. Stringent conditions may comprise low salt and/or high temperature conditions, such as provided by about 0.02 M to about 0.15 M NaCl at temperatures of about 40° C. to about 70° C. for oligonucleotides of 20-50 base pairs. It is understood that the temperature and ionic strength of a desired stringency are determined in part by the length of the particular nucleic acids, the length and nucleobase content of the target sequences, the charge composition of the nucleic acids, and the presence or concentration of formamide, tetramethylammonium chloride or other solvents in a hybridization mixture.

It is also understood that the ranges, compositions and conditions for hybridization are mentioned by way of non-limiting examples only, and that the desired stringency for a particular hybridization reaction is often determined empirically by comparison to one or more positive or negative controls. Non-limiting examples of low stringency conditions include hybridization performed at about 0.15 M to about 0.9 M NaCl at a temperature range of about 20° C. to about 50° C. Of course, it is within the skill of one in the art to further modify the low or high stringency conditions to suite a particular application.

Nucleic acids in the context of this invention include “oligonucleotides,” which refers to an oligomer or polymer of ribonucleic acid (RNA) or deoxyribonucleic acid (DNA) or chimeras of both. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides preferred over native forms to obtain enhanced affinity for the nucleic acid target and increased stability in the presence of nucleases.

A further preferred modification includes oligonucleotide primers or probes that include Locked Nucleic Acids (LNAs) in which the 2′-hydroxyl group is linked to the 3′ or 4′ carbon atom of the sugar ring thereby forming a bicyclic sugar moiety. The linkage is preferably a methelyne (—CH₂—)ₙ group bridging the 2′ oxygen atom and the 4′ carbon atom wherein n is 1 or 2. LNAs and preparation thereof are described in WO 98/39352 and WO 99/14226.

Other modifications include 2′-methoxy(2′—O—CH₃), 2′-aminopropoxy (2′—OCH₂CH₂CH₂NH₂), 2′-allyl (2′—CH₂—CH═CH₂), 2′-O-allyl (2′O—CH₂—CH═CH₂) and 2′-fluoro(2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine. (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, and other alkynyl derivatives of pyrimidine bases, 6-azo uracil, cytosine and thymine, 5-uracil(pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941.

### Cartridge-Based Analysis and Hand-Held Portable Nucleic Acid Detection Devices

The present invention also includes self-contained nucleotide-based bioanalytical systems incorporated into portable devices that incorporate the polymer particles or other particles for nucleotide capture, detection, and quantitation using the methods of the present invention. Advances in system miniaturization make it possible to combine sample preparation, amplification and detection in a single portable device that may include cartridges containing chambers, channels, and/or heating or cooling elements to store and manipulate samples and reagents and/or scan particles as described for various embodiments of the multiplexed end point quantitative PCR assays. Any of the components and procedures disclosed herein may be combined with cartridge-based analysis. In certain embodiments the cartridges include chambers containing one or more primer pairs for priming amplification of one or more corresponding nucleic acid targets, preferably pre-labeled with fluorescent labels, and one or more particles on which are immobilized a plurality of probes complementary to a corresponding nucleic acid targets, preferably encoded hydrogel particles. The cartridges may contain heating or cooling elements to perform amplification, as well as flow-focusing channels to align and flow particles for analysis. The cartridges are to be interfaced with equipment bearing active elements to deliver samples, manipulate flows, provide direct or indirect heating and cooling, illuminate the sample, and acquire fluorescent or other emitted signal. Cartridges may be disposable or reusable.

Hand-held or portable devices can be used in point-of-care facilities such as doctor's offices or hospitals, veterinarian's offices, pharmacies, diagnostics labs, and clinics; and also for detecting biological threats in civilian or military areas. A review of portable nucleic acid bioanalytical systems is provided by T. M. Lee et al., DNA-based bioanalytical microsystems for handheld device applications, Analytica Chemica, Acta 556 (2006) 26-37; see also T. Ray, UK Startup DNA Electronics Developing Handheld Device to Detect Genetic Risk for Drug AEs, Pharmacogenomics Reporter—Feb. 25, 2009; and K. P. O'Connell et al., Testing of the Bio-Seeq (Smiths Detection Handheld PCR Instrument): Sensitivity, Specificity, and Effect of Interferents on Bacillus Assay Performance, Edgewood Chemical Biological Center Aberdeen Proving Ground, MD Report No. A597724. The devices are suitable also for use in use in non-healthcare industries like food preparation, agriculture, and animal farming. The devices are preferably small in size (<8″ in all dimensions) and perform the assays described herein in less than two hours from the moment a sample is introduced. The devices contain active elements such as pumps, valves, optics, detectors, and electronics to introduce, manipulate, and interrogate samples. In certain embodiments, the devices may be used with cartridges or kits. In certain embodiments, the devices may contain reservoirs containing specific reagents including prelabeled primers, particles on which are immobilized one or more probes that detect amplicons of the targeted nucleic acids, PCR reagents, etc. as described herein. The devices may provide the capability to analyze one or multiple samples simultaneously.

### EXAMPLES

### Example 1

### Encoded Hydrogel Particle Sensitivity, Specificity and Thermal Stability

In order to show that the optimized particle design and labeling scheme did not negatively affect specificity, cross-reactivity of an RNA target with closely-related complementary probes was assessed using four different microRNAs from the let-7 family that varied by only one or two nucleotides in sequence as has been done extensively in the literature. Wang, H. et al., RNA-A Publication of the RNA Society 2007, 13, 151-159; 38; Lu, J, et al., Nature 2005, 435, 834-838; 39; and Chen, C.; et al., J. Nucleic Acids Res 2005, 33, e179.

Particles bearing four unique probe regions were synthesized, each probe region containing a unique probe for each of the four let-7 family members (7a-7d), which vary by only one or two nucleotides in sequence. The probes were incorporated at a precursor concentration of 10 μM. Particles were incubated with samples containing 5 femtomoles of biotinylated let-7a RNA and 500 ng of total E. coli RNA to add complexity, thus mimicking a “real” assay that would likely involve total human RNA consisting of broadly heterogeneous nucleic acid mixtures. Incubations were one hour at 58° C. with 0.5M NaCl in the hybridization buffer.

The results showed that multiplexed hydrogel encoded particles provided single-nucleotide specificity with less than 3% cross-reactivity (FIG. 1) without the use of locked nucleic acids or altered probe design. These initial results showed that one chamber multiplexed quantitative end point PCR is well-suited to meet the demands of clinical diagnostics for SNPs. Also shown in the figure is a sensitivity chart for the predicted sensitivity found using kinetic models developed for Hydrogel encoded particles. The assay was also highly sensitive, providing sub-attomole sensitivity that far exceeds the ˜100 attomole sensitivity reported for the current state-of-the art bead-based system, Luminex™, even with shorter incubations.

### Thermal Stability of the Encoded Polymer Particles

Encoded hydrogel particles need to be stable upon heating to withstand the high-temperature demands of PCR reaction. The results of experiments shown in (FIG. 2) verify that target hybridization signal is identical with and without extensive heating. This experiment also demonstrates the sensitive/specific quantification of fluorescent target without post-hybridization rinsing, which is important for a “one-pot” assay. Particles bearing two probe regions, a negative control region, and barcode region were heated at 95° C. for 20 minutes. They were then incubated with 500 attomole target at 37° C. for 60 minutes and imaged immediately afterward. Particles subjected to heating gave similar fluorescent signal as those which were not heated. No rinsing steps were required for this assay. The stability of the hydrogel particles at 95° C. for 20 minutes was not a foregone conclusion since the particles have relatively high water content and could have either deformed so that they could not be readily scanned, or sensitivity could have been reduced.

Presently particles can be made with about 3,000 different unique identification codes. Multiplex encoded polymer particles were synthesized using flow lithography, and are described at length in US. Serial No. US2007/0105972, and US 2008/0176216, Pregibon, D. C., M. Toner, and P. S. Doyle, Multifunctional encoded particles for high-throughput biomolecule analysis. Science, 2007. 315(5817): p. 1393-1396, Pregibon, D. C. and P. S. Doyle, Optimization of Encoded Hydrogel Particles for Nucleic Acid Analytical Chemistry, Vol. 81, No. 12, Jun. 15, 2009, 4873-4881, incorporated herein by reference.

### Example 2

### Multiplexed End Point-PCR Amplification and Detection of Two Unique Targets on λ-Phage DNA

λ-phage DNA target sequences were selected that were 60 base pairs long and primers were selected that had no significant homology to homo sapiens in a BLAST search. The sense strand of the λ-phage DNA was amplified, therefore the reverse primer was designed to incorporate a 5′ Cy3 modification for fluorescent detection of the amplicons.

Forward and reverse PCR primers (SEQ ID NOS: 1 and 2, respectively) twenty base pairs long were designed with a melting temperature Tₘ near 55° C. and minimal hairpin formation or interaction. The reverse primer did not have sufficient complementary to the probe in order to avoid nonspecific binding and extension. Probe sequences SEQ ID NOS: 3 and 4 were designed that (1) were complementary to a 40 base pair portion of the target sequence, (2) had a melting temperature Tₘ above 72° C., and (3) were unable to form stable hybrids with the labeled primer. The probe length was selected so that it formed a stable hybrid molecule with the amplicon at the 63° C. hybridization temperature. Each probe was incorporated into the appropriate probe region of a barcoded hydrogel particle. Details of the Primer and probe sequences are given below:

TABLE 3

| Nucleotide: | Sequence (5′ to 3′): | SEQ ID NO: |
|---|---|---|
| Probe #1 | /5Acryd/TAT CAT CAA | SEQ ID NO: 1 = |
| | AGC CAT GAA CAA AGC | the underlined |
| | AGC CGC GCT GGA TGA | sequence in the |
| | A/3Phos/ | second column |
| Forward #1 | TAT CAT CAA AGC CAT | SEQ ID NO: 2 |
| | GAA CA | |
| Reverse #1 | /5Cy3/TAT ATT CAC | SEQ ID NO: 3 = |
| | TCA GCA ACC CC | the underlined |
| | | sequence in the |
| | | second column |
| Probe #2 | /5Acryd/GAG TTC GTG | SEQ ID NO: 4 = |
| | TCC GTA CAA CTG GCG | the underlined |
| | TAA TCA TGG CCC TTC | sequence in the |
| | G/3Phos/ | second column |
| Forward #2 | GAG TTC GTG TCC GTA | SEQ ID NO: 5 |
| | CAA CT | |
| Reverse #2 | /5Cy3/GAC TCC TCC | SEQ ID NO: 6 |
| | ACA GAG AAA CA | |

This experiment was done with asymmetric primer concentrations to exhaust labeled primer and optimize the amount of labeled amplicons.

A single type of hydrogel particle was designed for this study, with rhodamine-methacrylate (Polysciences) for visualization of the encoded region, an inert negative control region flanking the encoded region, that in turn was flanked by two probe regions: one probe region containing probe #1 specific for amplicon #1, and the second probe region at the end of the particle with probe #2 specific for amplicon #2. Four experimental conditions were investigated: Primers for only target 1, Primers for both target 1 and target 2, Primers for only target 2 primers, and a control with neither of the primer sets.

PCR amplification and hybridization was done in a single tube in a 100 μL volume using: Taq polymerase (1×, diluted from 5× X master mix—New England Biolabs™), approximately 10⁴ copies of whole λ-phage DNA, forward primer at a final concentration of 0.2 μM, Cy3-labeled reverse primer at a final concentration of 0.4 μM, a final concentration of 0.05% tween, and approximately 100 barcoded hydrogel particles. Note that 100 particles for this volume assay was used to ensure there were excess particles for ease of finding and scanning them; however, as few as about 10 particles is adequate; 30 particles is preferred. A relatively high amount of target (10⁴ copies of whole λ-phage DNA) was used together with an excess of particles to optimize the signal. Hydrogel particle probe regions typically contain about 5 μM DNA probe covalently linked throughout the PEG hydrogel matrix. A total of 30 PCR amplification cycles were used.

The initial PCR denaturation was 5 minutes at 95° C. followed by 30 cycles of amplification that included a 30 second 95° C. denaturation, 30 second 49° C. primer annealing, and 45 second 72° C. extension. A final extension of 5 minutes at 72° C. occurred prior to a 1 minute 95° C. denaturation. Finally the sample was cooled to the probe hybridization temperature of 63° C. to promote stringent hybridization. Hybridization was promoted by shaking at 1800 rpm while maintaining a temperature of 63° C. for one hour using a thermomixer™ (Rio, Quantafoil). Note that although a primer annealing temperature of 49° C. was used in these experiments for the primer with a melting temperature of 55° C., a higher primer annealing temperatures could have been used, for example up to about 55° C. The user can vary the temperatures using routine experimentation to optimize specificity.

After hybridization, the samples (that now included labeled amplicons #1 and #2 hybridized to respective probes #1 and #2 at known sites on the encoded hydrogel particles) were diluted 1:4 in PTET buffer (5× TE with 25% PEG (n=400)) and 0.05% Tween 20. Particles were directly imaged using a fluorescence microscope and EB-CCD camera. There is no requirement for washing the particles prior to scanning with the present methods.

These experiments with individual Cy3-labeled primers for target #1 or target #2 λ-phage DNA showed that amplification and detection of the intended DNA targets using the new one chamber multiplexed quantitative end point PCR method with a hybridization temperature that was significantly higher (14° C.) than the primer annealing temperature was both highly sensitive and specific. Although amplification of target 1 appeared to be much more efficient than that of target 2, the efficiency of amplification of target 2 can be increased with routine experimentation by optimizing the design of probe #2, optimizing primer concentrations, or using more PCR cycles.

### Example 3

### Cell Culture and RNA Isolation

Experiments showing the specificity, sensitivity and quantitation of target mRNAs amplified by PCR and detected using encoded hydrogel particles are described.

Similar to the protocol described in Peck et al., Genome Biology, 7:R61, 2006, HL60 (human promyelocytic leukemia) cells were cultured in RPMI supplemented with 10% fetal bovine serum and antibiotics. Cells were treated with 1 μmol/l tretinoin (all-trans retinoic acid; Sigma-Aldrich, St Louis, Mo., USA) in dimethyl sulfoxide (DMSO; final concentration 0.1%) or DMSO alone for five days. Total RNA was isolated from bulk cultures with TRIzol Reagent (Invitrogen, Carlsbad, Calif., USA), in accordance with the manufacturer's directions. For the classification exercise, microtiter plate cultures were treated with 200 nmol/l tretinoin or DMSO for two days to mimic the submaximal signatures likely to be encountered in a small molecule screen, and were and prepared for mRNA capture by the addition of Lysis Buffer (RNAture, Irvine, Calif., USA).

### Probes and Primers

Similar to the protocol described in Peck et al., Genome Biology, 7:R61, 2006, upstream probes were composed (5′ to 3′) of the complement of the T7 primer site (SEQ ID NO: 7: TAA TAC GAC TCA CTA TAG GG), a 24 nucleotide (nt) barcode, and a 20 nucleotide gene-specific sequence. Downstream probes were 5′-phosphorylated, and contained a 20 nucleotide gene-specific sequence and the T3 primer site (SEQ ID NO: 8: TCC CTT TAG TGA GGG TTA AT). Barcode sequences were developed by Tm Bioscience (Toronto, Ontarion, Canada) and detailed in the Luminex F1exMAP Microspheres Product Information Sheet. Gene-specific fragments of probes were designed against the Oligator Human Genome RefSet, keyed by RefSeq identifier, where available. A 40 nucleotide region was manually selected from within these 70 nucleotide sequences to yield two fragments of equal length with roughly similar base composition and juxtaposing nucleotides being C-G or G-C, where possible. Probe sequences are provided in Additional data file 2 of [Peck et al., Genome Biology, 7:R61, 2006]. Capture probes contained the complement of the barcode sequences and had 5′-amino modification and a C12 linker. The T7 primer (SEQ ID NO: 7: 5′-TAA TAC GAC TCA CTA TAG GG-3′) was 5′-biotinylated. The T3 primer has SEQ ID NO: 9: 5′-ATT AAC CCT CAC TAA AGG GA-3′. Oligonucleotides (all with standard desalting) were from Integrated DNA Technologies (Coralville, Iowa, USA).

TABLE 4

| RefSeq | RefSet | FlexMAP | capture probe | upstream |
|---|---|---|---|---|
| ID | ID | ID | sequence | probe sequence |
| NM_000962 | HG_010_04807 | LUA#7 | ATTGGTAAATTG | TAATACGACTCACTATAGGGCA |
| | | | GTAAATGAATTG | ATTCATTTACCAATTTACCAAT |
| | | | SEQ ID | ACTCCTGCCTGAGTTTCCAG |
| | | | NO: 10 | SEQ ID NO: 11 |
| NM_006432 | HG_010_08035 | LUA#27 | AAAGTTGAGTAT | TAATACGACTCACTATAGGGCT |
| | | | TGATTTGAAAAG | TTTCAAATCAATACTCAACTTT |
| | | | SEQ ID | CAGAAACTGAGCTCCGGGTG |
| | | | NO: 12 | SEQ ID NO: 13 |
| NM_004095 | HG_010_07678 | LUA#59 | AAAGTGAAAAAG | TAATACGACTCACTATAGGGTC |
| | | | ATTGATTGATGA | ATCAATCAATCTTTTTCACTTT |
| | | | SEQ ID | TCCTTAGGTTGATGTGCTTG |
| | | | NO: 14 | SEQ ID NO: 15 |
| NM_003132 | HG_010_17983 | LUA#68 | AAAGAAAGATTG | TAATACGACTCACTATAGGGTC |
| | | | TTGAGATTATGA | ATAATCTCAACAATCTTTCTTT |
| | | | SEQ ID | TCTGGCGTTCCACCTCCAAG |
| | | | NO: 16 | SEQ ID NO: 17 |
| NM_002046 | Description | LUA#95 | TTAGTGTAGTAA | TAATACGACTCACTATAGGGTA |
| | GAPDH3 | | GTTTAAAGTGTA | CACTTTAAACTTACTACACTAA |
| | | | SEQ ID | CCCTGGACCACCAGCCCCAG |
| | | | NO: 18 | SEQ ID NO: 19 |

### Beads and Bead Coupling for Luminex Assays

Similar to the protocol described in Peck et al., Genome Biology, 7:R61, 2006, Luminex xMAP Multi-Analyte COOH Microspheres were coupled to capture probes in a semi-automated microtiter plate format. Approximately 2.5×106 microspheres were dispensed to the wells of a V-bottomed microtiter plate, pelleted by centrifugation at 1800 g for 3 minutes, and the supernatant removed. Beads were resuspended in 25 μl binding buffer (0.1 M 2-[N-morpholino]ethansulfonic acid; pH 4.5) by sonication and pipeting, and 100 pmol capture probe was added. A volume of 2.5 μl of a freshly prepared 10 mg/ml aqueous solution of 1-ethyl-3-(3-dimethylaminopropyl)carbodiimide hydrochloride (Pierce, Milwaukee, Wis., USA) was added, and the plate incubated at room temperature in the dark for 30 minutes. This addition and incubation step was repeated, and 180 μl 0.02% Tween-20 added with mixing. Beads were pelleted by centrifugation, as before, and washed sequentially in 180 μl 0.1% sodium dodecyl sulfate and 180 μl tris-EDTA (TE) (pH 8.0) with intervening spins. Coupled microspheres were resuspended in 50 μl TE (pH 8.0) and stored in the dark at 4° C. for up to one month. Bead mixes were freshly prepared and contained about 1.5×105/ml of each microsphere in 1.5× TMAC buffer (4.5 mol/ltetramethylammonium chloride, 0.15% N-lauryl sarcosine, 75 mmol/1 tris-HCl [pH 8.0], and 6 mmol/l EDTA [pH 8.0]). The mapping of bead number to capture probe sequence is provided in Additional data file 3 of [Peck et al., Genome Biology, 7:R61, 2006].

### Ligation-Mediated Amplification

Similar to the protocol described in Peck et al., Genome Biology, 7:R61, 2006, transcripts were captured in oligo-dT coated 384 well plates (GenePlateHT; RNAture) from total RNA (500 ng) in Lysis Buffer (RNAture) or whole cell lysates (20 μl). Plates were covered and centrifuged at 500 g for one minute, and incubated at room temperature for one hour. Unbound material was removed by inverting the plate onto an absorbent towel and spinning as before. A volume of 5 μl of an M-MLV reverse transcriptase reaction mix (Promega, Madison, Wis., USA) containing 125 μmol/l of each dNTP (Invitrogen) was added. The plate was covered, spun as before, and incubated at 37° C. for 90 minutes. Wells were emptied by centrifugation, as before. A volume of 10 fmol of each probe was added in 1× Taq Ligase Buffer (New England Biolabs, Ipswich, Mass., USA; 5 μl), the plate covered, spun as before, heated at 95° C. for two minutes and maintained at 50° C. for six hours. Unannealed probes were removed by centrifugation, as before. A volume of 5 μl of 1× Taq Ligase Buffer containing 2.5 U Taq DNA ligase (New England Biolabs) was added, the plate covered, spun as before, and incubated at 45° C. for one hour followed by 65° C. for 10 minutes. Wells were emptied by centrifugation, as before. A volume of 15 μl of a HotStarTaq DNA Polymerase™ mix (Qiagen, Hilden, Germany) containing 16 μmol/l of each dNTP (Invitrogen) and 100 nmol/l of T3 primer and biotinylated T7 primer was added. The plate was covered, spun as before, and polymerase chain reaction performed in a Thermo Electron (Milford, Mass., USA) MBS 384 Satellite Thermal Cycler (initial denaturation of 92° C. for 9 minutes, 92° C. for 30 s, 60° C. for 30 s, 72° C. for 30 s for 39 cycles; final extension at 72° C. for 5 minutes). Total time from the addition of lysis buffer to hybridization-ready product (referred to herein as the “LMA reaction product”) for 96 samples processed in parallel in a single microtiter plate is approximately 14 hours.

### Hybridization and Detection for Luminex Assays

Similar to the protocol described in Peck et al., Genome Biology, 7:R61, 2006, a volume of 15 μl of LMA reaction product was mixed with 5 μl TE (pH 8.0) and 30 μl bead mix (about 4,500 of each microsphere) in the wells of a Thermowell™ P microtiter plate (Costar, Corning, N.Y., USA). The plate was covered and incubated at 95° C. for two minutes and maintained at 45° C. for 60 minutes. A volume of 20 μl of a reporter mix containing 10 ng/μl streptavidin R-phycoerythrin conjugate (Molecular Probes, Eugene, Oreg., USA) in 1× TMAC buffer (3 mol/l tetramethylammonium chloride, 0.1% N-lauryl sarcosine, 50 mmol/l tris-HCl [pH 8.0], 4 mmol/l EDTA [pH 8.0]) was added with mixing and incubation continued at 45° C. for five minutes. Beads were analyzed with a Luminex 100 instrument. Sample volume was set at 50 μl and flow rate was 60 μl/minute. A minimum of 100 events were recorded for each bead set and median fluorescence intensities (MFIs) computed. Total time from the start of hybridization to download of raw data from the instrument for 96 samples processed in parallel in a single microtiter plate is approximately three hours. Expression values for each transcript were corrected for background signal by subtracting the MFI of corresponding bead sets from blank (TE only) wells. Values below an arbitrary baseline (5) were set to 5, and all were normalized against an internal control feature (GAPDH3).

### Encoded Hydrogel Particle Synthesis Using Stop-Flow Lithography

Precursor solutions consisted of blends of poly(ethylene glycol) diacrylate [(PEG-DA, Mn=700), ˜70 cP at 25° C., Aldrich] and PEG (Mw ˜200, ˜50 cP at 25° C., Aldrich) in 35% 3× Tris-EDTA buffer (pH)) 8.0, EMD) with 5% Darocur 1173 photoinitiator (Alrich). When applicable, DNA probe modified with an acrydite group (IDT) was included in precursor solutions at concentrations of 100 μM. These precursor samples were loaded into channels using pipette tips (200 μL, Molecular BioProducts), connected with rubber tubing (Tygon™) to a common pressure source (regulated by a pressure valve, Controlair Inc.). The tips were filled with ˜25 μL of polymer and inserted into the channel inlet ports. A three-way solenoid valve (Burkert) allowed for the oscillation between pressurized (at ˜3 psi, high velocity) and ambient-pressure (no flow) states. A valving system with resistive elements (filter-top pipet tips, Molecular BioProducts) and needle valves (Swagelok) provided independent control of the stream widths. Visual alignment for polymerization was achieved using a CCD camera (KPM1A, Hitachi) with NIH Image software. Control of flow (via solenoid valve) and UV exposure doses was accomplished using a custom-written script in LabView™ to allow continuous synthesis of particles. Times for flow, hold, UV exposure, and hold were 500, 300, 75, and 125 ms, respectively.

### Hybridization and Detection for Encoded Hydrogel Particles

In 0.65 mL Eppendorf tubes, a volume of 15 μl of LMA reaction product was added to 5 ul of encoded particles (at a concentration of ˜10 particles/ul in Tris-EDTA buffer, pH=8) and 50 ul of hybridization buffer containing 0.5M NaCl and 0.05% Tween-20 in Tris-EDTA buffer, pH=8. Tubes were vortexed for 3 seconds and heated to 95C for 5 minutes in a water bath. Incubations were carried out at for 90 minutes at 45C in a water bath. Particles were rinsed two times in phosphate buffered saline containing 0.05% Tween-20 by adding 500 ul of rinse buffer to the tubes, centrifuging for 30 seconds, and aspirating ˜500 ul from the tube. Five microliters of the reporter streptavidin-phycoerythrin (Invitrogen), diluted 50× in PBS, was added to each tube and the tubes were incubated at 45C for 30 minutes in a water bath. Each tube was then rinsed two times with buffer containing a 3:1 mix of PBS and poly(ethylene glycol) 200 with 0.05% Tween-20. Rinsed particle samples were pipetted into glass slides and sealed with a cover slip, which was then mounted on a Zeiss Axiovert 200 microscope. NIH Image was used to visualize images captured from an EB-CCD camera (C7190-20, Hamamatsu) mounted to the side port of the microscope with camera settings of 6.6, 1.3, and 2.9 for gain, offset, and sensitivity, respectively. A Zeiss A-Plan 10× objective (NA) 0.25) and an Exfo X-Cite™ illumination source (series 120) was used at the highest setting. Movies taken in NIH Image at 20 frames/s over 10 frames were averaged and saved as a single image. These images were analyzed using ImageJ™ software.

### Example 4

### One Chamber Multiplexed Quantitative End Point PCR Detection of RNA Viruses in a Biological Sample

Detection of Respiratory Diseases such as Influenza a (h1N1) and RSV (such as A-2)

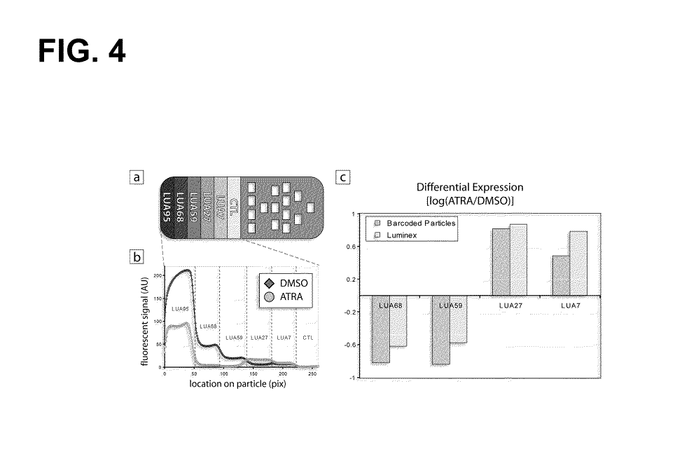

The one chamber multiplexed quantitative end point PCR assay can be adapted for amplifying and detecting RNA or DNA viruses, exemplified here using Human Influenza A (H1N1) and Respiratory Syncytial Virus (RSV) using the one chamber multiplexed quantitative end point PCR by following the basic steps below. A person of skill in the art will know how to vary or adapt these steps to a particular pathogen or specific goal. FIG. 4.

“Simulated samples” with human genomic DNA (available from Aviva Systems Biology), inactivated viral RNA for Influenza and RSV (both Advanced Biotechnologies), and purified Phage MS2 (DSMZ, Germany) as a control can be used for assay optimization. Note that if the pathogen were a DNA virus, the control would be for example lambda phage DNA).

Primer sets will be designed to make amplicons of pathogen-specific nucleic acid targets, each <100 base pairs in length modified with a single Cy3 fluorophore. Primer concentration, PCR cycles, and hybridization time on detection signal will be varied to optimize the assay ideally to achieve reproducible detection of ˜10 RNA templates per target nucleic acid. For RNA viruses, it is necessary to first produce cDNA libraries prior to PCR amplification. Stockton, J., et al., Multiplex PCR for typing and subtyping influenza and respiratory syncytial viruses. J Clin Microbiol, 1998. 36(10): p. 2990-5.

Potential primers sets as described herein will be identified for amplifying targeted pathogen nucleic acid using a primer-design program (such as Invitrogen's OligoPrimer™ for instance) to search through a user-input genomic sequence, identifying sets of primers that provide the desired primer melting temperatures and amplicon sizes. Ideal target regions of pathogen RNA are unique for the species and highly conserved. Each potential primer identified will be assessed for species-specificity via BLAST search. A script written in MATLAB or freely available software (like UNAfold), for example, can be used to assess dimer-formation with all other primers. Primers are preferably designed to have melting temperatures near 55° C., be ˜20 base pairs in length, and provide amplicons ˜60 base pairs in size. The primer incorporated into the amplicon is ideally prelabeled for example with a single Cy3 label for fluorescence detection,

Testing can be done using incubations with ˜30 particles (3 μl of particles at 10 particles/μl) pipetted into mPCR product for a total sample volume of 52 μl in 0.65 ml Eppendorf tubes. Standard incubations will be carried out typically at about 60° C. for one hour with rapid mixing (at 1800 rpm) in a thermomixer (Rio, Quantafoil). After hybridization, particles are scanned to determine binding of labeled amplicons to probes immobilized on the particles.

### Selection of Optimal Amplicon Targets

Once the basic parameters are established with Phage MS2 RNA, the assay will be run to amplify species-specific targets from Phage MS2 RNA, Influenza A and RSV genomic RNA, and use hybridization assays with all three particles types in a single sample.

### Determine Effect of PCR Cycles on Assay Sensitivity

Amplification via PCR is an exponential process. As such, in an assay the actual number of templates in the PCR reaction is not being quantified, instead the number captured on hydrogel particles is determined. By knowing the amplification efficiency of a given target along with the number of amplification cycles, the amount of amplicon captured on a particle can then be used to estimate the nucleic acid templates initially present. Using the optimized primer concentrations, the optimum number of PCR cycles for a known amount of target can be determined.

### Determine Effect of Hybridization Time on Assay Sensitivity

Hybridization time and temperature can be varied to find optimum conditions, starting for example using a hydrogel particle with a probe having a melting temperature of about 70 degrees and primers having a melting temperature of about 55 degrees. To calibrate, each test is repeated 10 times to assess reproducibility and specificity at varying pathogen concentrations. Using methods known in the art, (1) sensitivity, (2) specificity, (3) inter-run variance, and (4) run-to-run variance of the assays are determined and adjusted to optimum conditions.

Once the assay is optimized, clinically-relevant samples, including throat swabs and sputum specimens, will be analyzed.

The presence of PCR inhibitors (e.g. melanin) can be devastating to a nucleic acid amplification test. For this reason, tests are run in the presence of inhibitors to choose conditions to assure that the Multiplexed End-Point PCR assays reproducibly detect inhibition, including optimizing the concentration of the positive control to accurately reflect the presence of an inhibitor.

In the foregoing specification, the invention has been described with reference to specific embodiments. It will, however, be evident that various modifications and changes may be made without departing from the broader spirit and scope of the invention. The specification and drawings are, accordingly, to be regarded in an illustrative rather than a restrictive sense. The contents of all references, pending patent applications and published patents, cited throughout this application are hereby expressly incorporated by reference as if set forth herein in their entirety, except where terminology is not consistent with the definitions herein. Although specific terms are employed, they are used as in the art unless otherwise indicated.

What is claimed is:

1. A method of amplifying and quantifying a plurality of nucleic acid targets in a sample comprising:
a. combining in a chamber:
the sample comprising the plurality of nucleic acid targets;
a labeling agent,
a plurality of primer pairs for priming amplification of the plurality of nucleic acid targets, wherein the primer pairs hybridize to the targets at a primer annealing temperature,
a plurality of particles on which are immobilized a plurality of nucleic acid probes that are complementary to the plurality of nucleic acid targets,
and a PCR cocktail containing enzymes for amplifying the nucleic acid targets;
b. performing one or more amplification cycles to form labeled amplification products for each of the plurality of nucleic acid targets,
c. hybridizing the labeled amplification products for each of the plurality of nucleic acid targets to the respective complementary probes at a hybridization temperature that is at least from about 2-15° C. higher than the primer annealing temperature but lower than a Tm of target-probe complexes;
d. detecting and quantifying a signal from the labeled amplification products for each of the plurality of nucleic acid targets hybridized to the respective complementary probes; and
e. comparing the quantified amplification product signal for each of the nucleic acid targets to a signal from a known amount of a known reference nucleic acid to quantify the amount of each nucleic acid target in the sample.

2. The method of claim 1, wherein the labeling agent binds to one primer of each of the primer pairs.

3. The method of claim 1, wherein the mixture of step a further comprises a free probe that is complementary to a region of the amplification product and wherein the free probe is bound to the labeling agent.

4. The method of claim 1, wherein the labeling agent is a fluorescent label selected from the group comprising 6-FAM™, Alexa Fluor, Fluorescein, Phycoerythrin, Cy3, Cy5, Cy5.5, Dy 750, HEX™, Iowa Black®, IRDye®, Joe, LightCycler 640, MAX 550, Rhodamine Green™, Rhodamine Red™, ROX™, TET™, TEX 615, Texas Red®, TYE (including TYE™ 563, TYE™ 665, TYE™ 705), WellRED™ D2, WellRED™ D3, WellRED™ D4 and TAMRA dyes.

5. The method of claim 1, wherein the known reference nucleic acid of step e) is an endogenous reference gene, or an external nucleic acid added to the sample, or the nucleic acid target of which known amounts are plotted on a standard curve.

6. The method of claim 1, wherein the endogenous reference gene is a member selected from the group consisting of nucleolar RNA, beta-actin, GADPH or 18S RNA.

7. The method of claim 1, wherein the labeling agent is a radioisotope or quantum dots.

8. The method of claim 1, wherein the probe comprises a 10 base pair to about 50 base pair sequence that is complementary to the target nucleic acid sequence.

9. The method of claim 1, wherein the 3′ end of the probe comprises a blocked 3′ hydroxyl group.

10. The method of claim 9, wherein the 3′ hydroxyl group is blocked with a phosphate group or a 3′ inverted dT or dideoxycytidine modification.

11. The method of claim 1, wherein the probes comprise a locked nucleic acid (LNA) modification.

12. The method of claim 1, wherein the primer annealing temperature is from about 35° C. to about 60° C.

13. The method of claim 1, wherein the hybridization temperature is from about 37 to about 75° C.

14. The method of claim 1, wherein the primers are from about 10 to about 25 base pairs in length.

15. The method of claim 1, wherein the primers have a melting temperature of about 35° C. to about 65° C. and the probe-amplicon complexes have a melting temperature of about 40 to about 75° C.

16. The method of claim 1, wherein the hybridization step (c) is from about 20 minutes to about 90 minutes long.

17. The method of claim 1, wherein the amplification products are from about 50 to about 100 base pairs long, preferably from about 50 to about 70 base pairs long.

18. The method of claim 1, wherein the nucleic acid target is DNA and the enzymes comprise DNA polymerase.

19. The method of claim 1, wherein the nucleic acid target is RNA and the enzymes comprise DNA polymerase and reverse transcriptase.

20. The method of claim 1, wherein from about 20 to about 40 amplification cycles are performed in step (b).

21. The method of claim 1, wherein the particles are encoded particles comprising one or more different probes that are either immobilized on the particle or incorporated into the particle substrate.

22. The method of claim 21, wherein the particles are polymer particles comprising polyethylene glycol.

23. The method of claim 21 wherein the particles are encoded using spectrometric or graphical codes, radio frequencies, electronic or physical magnetic properties, radioactivity, or diffractive gratings.

24. The method of claim 21, wherein the particles are composed of glass, silica, or metal.

25. A method of amplifying and detecting genetic variation at a known mutation site in a gene in a sample comprising a plurality of nucleic acid targets comprising:
(a) combining in a chamber
a first primer pair for priming amplification of a first allele of the gene, wherein the primers hybridize to the targets at a primer annealing temperature, and wherein one primer of the first primer pair is labeled with a first reporter, and
a second primer pair for priming amplification of a second allele of the gene, wherein the primers hybridize to the targets at a primer annealing temperature, and wherein one primer of the first primer pair is labeled with a second reporter,
a plurality of particles on which are immobilized a plurality of nucleic acid probes that are complementary to a nucleic acid sequence that is common to both the first and second alleles, wherein the common sequence is adjacent to the known mutation site, and a PCR cocktail comprising enzymes for amplifying nucleic acid targets,
(b) performing one or more amplification cycles to form labeled amplification products for the first and second alleles,
(c) hybridizing the labeled amplification products to the probes at a hybridization temperature that is at least about 2° C. to 15° C. degrees higher than the primer annealing temperature, and
(d) detecting a signal from the reporters on the labeled amplification products hybridized to the probes on the particles, and comparing the two signals thereby detecting the relative quantities of the first and the second alleles on the particle.

26. The method of claim 25, wherein the genetic variability is a single nucleotide polymorphism.

27. The method of claim 25, wherein the genetic variability is a deletion or insertion.

28. The method of claim 25, further comprising adding up to four additional unique primer pairs in step a) for priming amplification of up to four additional alleles of the, wherein the respective primers hybridize to the respective targets at a primer annealing temperature, and wherein one primer of each additional unique primer pair is labeled with a unique reporter.

29. The method of claim 25, wherein the reporters are fluorophores selected from the group comprising 6-FAM™, Alexa Fluor, Fluorescein, Phycoerythrin, Cy3, Cy5, Cy5.5, Dy 750, HEX™, Iowa Black®, IRDye®, Joe, LightCycler 640, MAX 550, Rhodamine Green™, Rhodamine Red™, ROX™, TET™, TEX 615, Texas Red®, TYE™ 563, TYE™ 665, TYE™ 705, WellRED™ D2, WellRED™ D3, WellRED™ D4, and TAMRA dyes.

30. The method of claim 25, wherein the reporters are radioisotopes or quantum dots.

31. The method of claim 25, wherein the primer labeled with the first reporter comprises a nucleotide at the 3′ end that is complementary to the nucleotide in the first allele at the known mutation site, and the primer labeled with the second reporter comprises a nucleotide at the 3′ end that is complementary to the nucleotide in the second allele at the known mutation site.

32. A nucleic acid probe that is complementary to a nucleic acid sequence that is common to two different alleles of a particular gene, which sequence is adjacent to a known SNP mutation site on the gene.

33. A particle for nucleic acid detection, comprising the probe of claim 32.

34. The particle of claim 33, wherein the particle is an encoded hydrogel particle.

35. A kit for detecting genetic variation at a known mutation site in a gene,
a first allele-specific primer pair for priming amplification of a first allele of the gene, wherein the primer that is extended to form the amplification product is labeled with a first reporter, and
a second allele-specific primer pair for priming amplification of a second allele of the gene, wherein the primer that is extended to form the amplification product is labeled with a second reporter, and
encoded hydrogel particles on which are immobilized a plurality of nucleic acid probes that are complementary to a nucleic acid sequence that is common to both the first and second alleles, wherein the common sequence is adjacent to the known mutation site.

36. The kit of claim 35, wherein the reporters are fluorophores selected from the group comprising 6-FAM™, Alexa Fluor, Fluorescein, Phycoerythrin, Cy3, Cy5, Cy5.5, Dy 750, HEX™, Iowa Black®, IRDye®, Joe, LightCycler 640, MAX 550, Rhodamine Green™, Rhodamine Red™, ROX™, TET™, TEX 615, Texas Red®, TYE™ 563, TYE™ 665, TYE™ 705, WellRED™ D2, WellRED™ D3, WellRED™ D4, and TAMRA dyes.

37. The method of claim 35, wherein the reporters are radioisotopes or quantum dots.

38. A cartridge for use in a portable device that performs multiplexed end-point quantitative PCR according to the steps of claim 1.

39. A portable device that that performs multiplexed end-point quantitative PCR according to the steps of claim 1.

40. The method of claim 21, wherein the particle comprises a fluorescently-labeled encoded region and one or more probe regions.
